SEQUENCE LISTING (4 sequences)

SEQ ID NO: 1 - protein, 237 aa, Artificial Sequence
  Fn14/TRAIL fusion protein
MRALLARLLLCVLVVSDSKGEQAPGTAPCSRGSSWSADLDKCMDCASCRARPHSDFCLGCAAAPPAPFRLLWRGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 2 - protein, 20 aa, Homo sapiens
MRALLARLLLCVLVVSDSKG

SEQ ID NO: 3 - protein, 52 aa, Homo sapiens
EQAPGTAPCSRGSSWSADLDKCMDCASCRARPHSDFCLGCAAAPPAPFRLLW

SEQ ID NO: 4 - protein, 165 aa, Homo sapiens
RGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

(19) United States
(12) Patent Application Publication
(10) Pub. No.: US 2018/0169183 A1
(43) Pub. Date: Jun. 21, 2018

(54) DUAL SIGNALING PROTEIN (DSP) FUSION PROTEINS, AND METHODS OF USING THEREOF FOR TREATING DISEASES

(71) Applicant: KAHR MEDICAL (2005) LTD, Jerusalem (IL)

(72) Inventors: Michal DRANITZKI ELHALEL, Shoresh (IL); Noam SHANI, Zichron Ya'akov (IL)

(73) Assignee: KAHR MEDICAL LTD, Jerusalem (IL)

(21) Appl. No.: 15/554,732

(22) PCT Filed: Mar. 3, 2016
(86) PCT No.: PCT/IL16/50244

§ 371 Date: Aug. 31, 2017

Related U.S. Application Data
(60) Provisional application No. 62/127,320, filed on Mar. 3, 2015.

(51) Int. Cl. A61K 38/17; A61P 35/00

(52) U.S. Cl. CPC A61K 38/177; A61P 35/00

(57) ABSTRACT

The invention provides a composition and use of a composition comprising a fusion protein and a first ligand in a ratio sufficient to increase therapeutic efficacy of the fusion protein in a subject, the fusion protein comprising an extracellular domain (ECD) of a first receptor and a second ligand, wherein the second ligand is capable of binding to a second receptor, wherein the first ligand is capable of binding to the first receptor, wherein the first and second ligands, and the first and second receptors, are TNF-family members, and wherein the first and second ligands are different, and the first and second receptors are different. The invention also provides a composition comprising Fn14-TRAIL fusion protein and TWEAK and use thereof for treating diseases or conditions associated with increased levels of TWEAK. The invention is further directed to use of Fn14-TRAIL fusion protein to treat diseases or conditions associated with increased levels of TWEAK.

### BACKGROUND OF THE INVENTION

Dual Signaling Proteins (DSP), also known as Signal-Converting-Proteins (SCP) which are currently known in the art are bi-functional fusion proteins that link an extracellular portion of a type I membrane protein (extracellular amino-terminus), to an extracellular portion of a type II membrane protein (extracellular carboxyl-terminus), forming a fusion protein with two active sides (see for example U.S. Pat. Nos. 7,569,663 and 8,039,437, both of which are hereby incorporated by reference as if fully set forth herein).

Fn14-TRAIL is a non-limiting example of such a DSP. TRAIL is a member of the Tumor Necrosis Factor (TNF) ligand superfamily and binds to a number of different cognate receptors of the TNF receptor superfamily, some leading to triggering of intracellular signaling pathways and others simply acting as decoy receptors. The triggering receptors in humans are TRAIL-R1 and TRAIL-R2, and in mice the sole triggering receptor is DR5. Virtually all cells of the immune system (T lymphocytes, B lymphocytes, natural killer cells, dendritic cells, monocytes, granulocytes) upregulate surface TRAIL and/or release soluble TRAIL stored in secretory vesicles in response to interferon and other activation signals. TRAIL inhibits autoimmunity in several animal models. Evidence for TRAIL'S capacity to inhibit experimental autoimmune encephalitis (EAE), a murine model for MS, has come from experiments invoking TRAIL−/− knockout mice, soluble TRAIL receptor (sDR5) or neutralizing anti-TRAIL mAb capable of blocking TRAIL function, and embryonic stem cell-derived dentritic cells co-expressing TRAIL and pathogenic MOG (myelin oligodendrocyte glycoprotein peptide). Interestingly, in MS patients, soluble TRAIL has emerged as a response marker for IFN-beta therapy, with those most likely to respond to treatment showing early and sustained soluble TRAIL induction after therapy. Yet, TRAIL'S impact on MS/EAE may be more complex, for example, the suggestion that TRAIL may promote brain cell apoptosis. Both TRAIL and FasL have been implicated in negative regulation of T cells.

TRAIL is known to be able to induce apoptosis of cancer cells, yet it has not been shown to be an effective anti-cancer therapy, despite attempts to use it as such (Lemke et al, “Getting TRAIL back on track for cancer therapy”, Cell Death and Differentiation (2014) 21, 1350-1364).

Fibroblast growth factor inducible 14 (Fn14, also known as TNF-like weak inducer of apoptosis receptor [TWEAK-R] or TNFRSF12A), is a member of the TNF receptor superfamily. Expression of Fn14 is up-regulated by growth factors in vitro and in vivo in response to tissue injury, regeneration, and inflammation. As one of the names for Fn14 suggests, this protein is a receptor for the protein designated TWEAK. TWEAK binding to Fn14, or constitutive Fn14 overexpression, activates the NFκB signaling pathway, which is known to play an important role in immune and inflammatory processes, oncogenesis, and cancer therapy resistance. This interaction also controls many cellular activities including, proliferation, migration, differentiation, apoptosis, angiogenesis and inflammation. TWEAK and Fn14 are also involved in tissue repair and regulation of immune functions and tumor growth and metastasis. Accordingly, Fn14-mediated signaling is involved in pathways that play important roles in human diseases. Fn14-mediated signaling has been suggested to play a role in numerous diseases, including, cancer, metastasis, immunological disorders (including autoimmune diseases, graft rejection and graft versus host disease, and chronic and acute neurological conditions [including stroke]).

Fn14 is expressed by many non-lymphoid cell types (epithelial, mesenchymal, endothelial cells and neurons), by many tissue progenitor cells, including all progenitor cells of the mesenchymal lineage. This protein is highly inducible by growth factors e.g., in serum that are encountered in vivo at sites of tissue injuries and/or tissue remodeling. As a consequence Fn14 expression is relatively low in most healthy tissues, but increased in injured and/or diseased tissues.

TWEAK protein is a cytokine that belongs to the TNF ligand superfamily. TWEAK mostly functions as a growth factor (mainly seen in cancer) and as an immune-inducer with overlapping signaling functions with TNF, but in specific scenarios it can also induce apoptosis via multiple pathways of cell death in a cell type-specific manner, or to promote proliferation and migration of endothelial cells, and thus acts as a regulator of angiogenesis.

TWEAK promotes the proliferation of some cell types (astrocytes, endothelial cells, and certain human tumor cell lines), and suppresses others (erythroblasts, kidney cells, mesangial cells, neuronal cells, NK cells, monocytes). TWEAK stimulates production of various inflammatory cytokines, chemokines and adhesion molecules. In addition, TWEAK increases the permeability of the neurovascular unit, and its endogenous expression is elevated in the CNS during Experimental Autoimmune Encephalomyelitis (EAE), a well-established model for the human disease Multiple Sclerosis (MS) and in acute cerebral ischemia. Moreover, TWEAK has pro-angiogenic activity, which is of interest given the association between angiogenesis and both cancer and autoimmune pathogenesis. TWEAK increases EAE severity in animal models and associated neurodegeneration.

### SUMMARY OF THE INVENTION

It is known in the art to provide certain combinations of TNF-family ligands and TNF-family receptors as fusion proteins, as described for example in U.S. Pat. Nos. 8,039,437 and 7,569,663.

Surprisingly, the present inventors have found that specific fusion proteins comprising an extracellular domain (ECD) of a first TNF family receptor and a second TNF-family ligand that may be advantageously administered to subjects suffering from inflammatory, immune related or cancerous diseases, depending upon the presence of a first TNF-family ligand, a second TNF-family receptor. In some embodiments, the second ligand is capable of binding to a second receptor, the first receptor is capable of binding to a first ligand, wherein the first and second ligands, and the first and second receptors or the ECD thereof, are TNF-family members, and wherein the first and second ligands are different, and the first and second receptors are different. Without wishing to be limited by a single hypothesis, the activity of the fusion protein may optionally be potentiated by the first TNF-family ligand, the second TNF-family receptor or the ECD thereof. Such other first TNF-family ligand or second TNF-family receptor may also optionally be administered with the fusion protein, as part of a stabilized composition.

The terms “DSP” and “fusion protein” are used herein interchangeably.

The present inventors have found that this combination of a fusion protein as described herein, plus an additional TNF-family ligand, may optionally be selected as follows. Consider two TNF-family ligand/receptor pairs, labeled A/B and C/D, respectively. A and C are the ligands or the ECD thereof; B and D are the receptors or the ECD thereof. It should be noted that the terms “ligand” and “receptor” are used only to describe these different combinations, as with regard to the TNF-family at least, it is known that receptors can be soluble and ligands can be attached to the cell membrane. Therefore, each of A, B, C and D may optionally be separately membrane-attached or soluble. The fusion protein, in some embodiments, comprises the ECD of each of the TNF-family ligand and the TNF-family receptor.

The receptors or the ECD thereof are different from each other, as are the ligands or the ECD thereof. Furthermore, ligand A does not bind to receptor D, nor does ligand C bind to receptor B. A fusion protein may optionally be constructed from the ECD (extracellular domain) of receptor B and ligand C. According to at least some embodiments, a diagnostic method to determine whether this fusion protein may be advantageously administered to a subject would therefore comprise detecting a level of ligand A in the subject, for example and without limitation globally (for example in a blood, Cerebrospinal Fluid (CSF), synovial fluid, saliva, or urine or any other body fluids or excretions) or locally, for example by testing specific diseased tissues or organs or the environment in the area of those diseased tissues or organs.

According to at least some embodiments, a stabilized composition may optionally comprise the above fusion protein, comprising the ECDs of receptor B and ligand C, plus a stabilizing amount of ligand A, or receptor D.

A non-limiting example of such a group of TNF-family ligand/receptor pairs is TWEAK (ligand A)/Fn14 (ECD of receptor B) and TRAIL (ECD of ligand C)/TRAIL-receptor (receptor D). Table 1 provided below shows TNF-family ligand and their receptor pairs, from which second ligand and a first receptor or an ECD thereof may be selected for the fusion protein and a first ligand or a second receptor or ECD thereof may be selected for adding into a composition comprising the fusion protein or for determining the presence thereof in a disease or condition for selecting the fusion protein to be used in a treatment of the disease or the condition with the proviso that the second ligand is capable of binding to a second receptor, the first receptor is capable of binding to a first ligand, wherein the first and second ligands are different, and the first and second receptors or the ECD thereof are different

TABLE 1
List of TNF receptors and ligands

| Ligand | Receptor |
|---|---|
| 4-1BBL | 4-1BB |
| APRIL | BCMA |
| BAFF | TACI |
| CD27L | CD27 |
| CD30L | CD30 |
| CD40L | CD40 |
| EDA-A1 | EDAR |
| EDA-A2 | XEDAR |
| TRAIL | TRAIL-R1 |
| | TRAIL-R2 |
| | TRAIL-R3 |
| | TRAIL-R4 |
| TRANCE/RANKL | OPG |
| | RANK |
| Unknown Lig. | TROY |
| FasL | Fas |
| GITRL | GITR |
| LIGHT | DcR3 |
| | HVEM |
| TL1A/VEGI | DR3 |
| TWEAK | TWEAKR/Fn14 |
| TNF-alpha | TNFR1 |
| TNF-beta | TNFR2 |
| Different lymphotoxins, such as LIGHT, | Lymphotoxin beta R |
| lymphotoxin alpha (LTA) and lymphotoxin | (LTBR) |
| beta (LTB) | |
| OX40L | OX40R |
| Neurotrophins, such as NGF and NTF4 | NGFR |
| APP | DR6 |
| Unknown Lig. | RELT |

Without wishing to be limited by a single hypothesis, it is possible that such an advantageous method of treatment and/or stabilized composition may be achieved for the following reasons. A fusion protein comprising receptor B and ligand C (for example, Fn14-TRAIL) is expected to trimerize due to trimerization of the ligand C (TRAIL). Upon administration and/or as part of a stabilized composition, and/or as present already in a diseased tissue or organ of the subject or in the environment of the diseased organ or tissue, the ligand A (TWEAK) will also trimerize and will bind receptor B (the ECD of Fn14) of three trimerized fusion proteins. FIGS. 16A-16C show an illustrative, non-limiting example of such trimerization and also oligomerization. These “trimers of trimers” will form a cluster. Again, without wishing to be limited by a single hypothesis, such a cluster would be expected to both block the activity of the ligand A (TWEAK) and to cluster the trimers of ligand C (TRAIL) and thereby potentiate TRAIL activity. This is exemplified in FIG. 16C showing that cluster of (A)+(BC) would likely form at locations in which (A) concentrations are high, for example, in the body if (A) is presented in higher amount in a diseased tissue or organ or their environment as compared to a healthy organ or tissue, the cluster will block the activity of (A) and induce the activity of (D) receptors on cell membranes via clustering of the (C) ligands, or the positioning the (C) ligands in a way that will enhance their binding to (D) receptors.

Accordingly, in some embodiments there is provided a complex comprising a fusion protein and a first ligand, the fusion protein comprising an extracellular domain (ECD) of a first receptor and a second ligand, wherein the second ligand is capable of binding to a second receptor, wherein the first ligand is capable of binding to the first receptor, wherein the first and second ligands, and the first and second receptors, are TNF-family members, and wherein the first and second ligands are different, and the first and second receptors are different. Optionally according to at least some embodiments, a diagnostic method that additionally or alternatively detects the presence of ligand “A” and/or receptor “D” may be provided, to determine whether to administer the above fusion protein, featuring the ECD of receptor “B” and ligand “C”, to the subject.

In some embodiments of the invention, the fusion protein comprising the ECDs of receptor “B” and ligand “C” according to the embodiments of the invention is administered to a subject having disease or condition that is associated with detectable or elevated levels of ligand “A” and/or receptor “D” as measured in the diseased organ, tissue, or in their environment, or in the blood, Cerebrospinal Fluid (CSF), synovial fluid, saliva, or urine or any other body fluids or excretions of a subject inflicted with such a disease or condition. The term “detectable” is in comparison to a healthy subject in which the presence of ligand “A” and/or receptor “D” cannot be detected in the same tissue, organ or in their environment or in the blood, Cerebrospinal Fluid (CSF), synovial fluid, saliva, or urine or in any other body fluids or excretions. The term “elevated levels” is also comparison to a healthy subject in which the level of ligand “A” and/or receptor “D” is lower than the level detected in the same tissue, organ or in their environment or in the blood, Cerebrospinal Fluid (CSF), synovial fluid, saliva, or urine or in any other body fluids or excretions by at least 20%.

According to at least some embodiments, there is provided a stabilized composition comprising a fusion protein and a first ligand in a ratio sufficient to increase therapeutic efficacy of the fusion protein in a subject, the fusion protein comprising an extracellular domain (ECD) of a first receptor and a second ligand, wherein the second ligand is capable of binding to a second receptor, wherein the first ligand is capable of binding to the first receptor, wherein the first and second ligands, and the first and second receptors, are TNF-family members, and wherein the first and second ligands are different, and the first and second receptors are different.

In this example, the fusion protein comprises the ECDs of receptor “B” (first receptor) and the second ligand (ligand “C”), and the composition further comprises the first ligand (ligand “A”).

Optionally increasing therapeutic efficacy comprises increasing half-life of the fusion protein in the subject as a non-limiting example.

According to at least some embodiments, there is provided a stabilized composition comprising a fusion protein and a first ligand in a ratio sufficient to increase migration time of the fusion protein in a native PAGE gel (comparing to the migration time of a composition comprising the fusion protein without the first ligand), the fusion protein comprising an extracellular domain (ECD) of a first receptor and a second ligand, wherein the second ligand is capable of binding to a second receptor, wherein the first ligand is capable of binding to the first receptor, wherein the first and second ligands, and the first and second receptors, are TNF-family members, and wherein the first and second ligands are different, and the first and second receptors are different.

According to at least some embodiments, there is provided a method of treating a disease in a subject, comprising: determining a level or a presence of a first ligand capable of binding to a first TNF-family receptor in a diseased organ, tissue or in their environment or in the blood, Cerebrospinal Fluid (CSF), synovial fluid, SALIVA, or urine or any other body fluids or excretions of the subject and/or determining a level or a presence of a second receptor capable of binding to a second TNF-family ligand a diseased organ, tissue or in their environment or in the blood or urine of the subject; if the level of the first ligand is above a baseline level, or if the first ligand or second receptor is not present in same tissue, organ or in their environment in the blood or urine of a healthy subject administering a fusion protein to the subject to treat the disease, the fusion protein comprising an extracellular domain (ECD) of the first receptor, wherein the fusion protein further comprises an ECD of a second TNF-family ligand, wherein the second TNF-family ligand is not capable of binding to the first receptor but wherein the second TNF-family ligand is capable of binding to a second TNF-family receptor, wherein the disease is treatable by activating the second TNF-family receptor.

By “baseline level” it is optionally meant a minimum level, which may optionally be any level above zero.

Optionally any composition as described herein may be administered with one of the above methods of treatment.

Optionally the method further comprises detecting a presence of a second TNF-family receptor, to which the second TNF-family ligand is capable of binding, in the subject; and if presented administering the fusion protein.

According to at least some embodiments, there is provided a method of treating a disease in a subject, comprising administering a fusion protein to the subject with a disease that is associated with detectable or elevated levels of ligand “A” and/or receptor “D” as measured in the diseased organ, tissue or in their environment or in the blood, Cerebrospinal Fluid (CSF), synovial fluid, SALIVA, or urine or any other body fluids or excretions of a subject inflicted with such a disease or condition, wherein the fusion protein comprising an extracellular domain (ECD) of the first receptor, wherein the fusion protein further comprises a second TNF-family ligand, wherein the second TNF-family ligand is not capable of binding to the first receptor but wherein the second TNF-family ligand is capable of binding to a second TNF-family receptor, wherein the disease is treatable by activating the second TNF-family receptor.

For example, if a disease is associated with elevated levels or is characterized by the presence of TWEAK in the diseased tissue, organ or in their environment, a fusion protein comprising the receptor of TWEAK (as Fn14) or its ECD is used. In a non limited example the fusion protein is Fn14-TRAIL.

By “Fn14-TRAIL fusion protein” it is meant a bi-component protein featuring a Fn14 domain and a TRAIL domain as described herein which are linked covalently. This fusion protein is also referred to herein as “Fn14-TRAIL”. Optionally and preferably, the bi-component protein comprises the extracellular domain of Fn14 and the extracellular domain of TRAIL. Optionally, the bi-component protein has an N-terminal side which is the extracellular domain of Fn14 and a C-terminal side which is composed of the extracellular domain of TRAIL.

A non-limiting example for diseases in which TWEAK is present or elevated in comparison to healthy subjects and that may be treatable by a fusion protein comprising Fn14 or the ECD thereof, such as for example, Fn14-TRAIL are: ovarian cancer, colorectal cancer, head and neck squamous cell carcinoma (HNSCC), colonic adenocarcinoma, hepatocellular carcinoma, kidney cancer, stomach cancer, breast cancer, squamous cell carcinoma, esophageal cancer, pancreatic cancer, cervical cancer, colorectal cancer, glioma, head and neck cancer, liver cancer, melanoma, prostate cancer, skin cancer, testis cancer, thyroid cancer, urothelial cancer, Hodgkin lymphoma metastatic neuroblastoma, glioblastoma, astrocytoma or astocytic brain tumor, lung carcinoma, pancreas adenocarcinoma, ovarian cystadenocarcinoma, cervical squamous carcinoma, prostate adenocarcinoma, squamous cell carcinoma, keratinocyte carcinoma, metastatic malignant melanoma, osteosarcoma, or metastatic choriocarcinoma

In some embodiments of the invention the diseases to be treated by a fusion protein comprising Fn14 or the ECD thereof, such as for example, Fn14-TRAIL are: seborrheic keratosis, systemic lupus erythematosus (SLE), Lupus Nephritis, Rheumatoid Arthritis (RA), inflammatory bowel disease (IBD) as ulcerative colitis and Crohn's disease, multiple sclerosis, Parkinson's disease, amyotrophic lateral sclerosis (ALS), atopic dermatitis, psoriasis vulgaris, psoriatic arthritis, urticarial vasculitis, myocardial infarction, proliferative diabetic retinopathy, or acute ischemic stroke

In some embodiments of the invention the diseases to be treated by a fusion protein comprising Fn14 or the ECD thereof, such as for example, Fn14-TRAIL are: seborrheic keratosis, Inflammatory bowel disease (IBD) as ulcerative colitis and Crohn's disease, Lupus Nephritis, Parkinson's disease, amyotrophic lateral sclerosis (ALS), psoriasis vulgaris, psoriatic arthritis, myocardial infarction, proliferative diabetic retinopathy, or retinopathy caused by any other condition (for example, hypertension, radiation, sickle cell disease and the like), or acute ischemic stroke.

In some embodiments of the invention the diseases to be treated by a fusion protein comprising Fn14 or the ECD thereof, such as for example, Fn14-TRAIL are: testis cancer, urothelial cancer, Hodgkin lymphoma, squamous cell carcinoma, keratinocyte carcinoma or osteosarcoma.

As described above, in Table 1 list of ligand-receptor pairs was provided. Such ligand-receptors may optionally be the first ligand/receptor pair (that is, ligand A and receptor B) or the second ligand/receptor pair (that is ligand C and receptor D), as long as ligand A and ligand C are different, and receptor B and receptor D are different.

Optionally, if the first receptor is 4-1BB, the first ligand is 4-1BBL; or alternatively if the second receptor is 4-1BB, the second ligand is 4-1BBL.

Optionally, if the first receptor is BCMA, the first ligand is APRIL or BAFF; or alternatively if the second receptor is BCMA, the second ligand is APRIL or BAFF.

Optionally, if the first receptor is CD27, the first ligand is CD27L; or alternatively if the second receptor is CD27, the second ligand is CD27L.

Optionally, if the first receptor is CD30, the first ligand is CD30L; or alternatively if the second receptor is CD30, the second ligand is CD30L.

Optionally, if the first receptor is CD40, the first ligand is CD40L; or alternatively if the second receptor is CD40, the second ligand is CD40L.

Optionally, if the first receptor is EDAR, the first ligand is EDA-A1; or alternatively if the second receptor is EDAR, the second ligand is EDA-A1.

Optionally, if the first receptor is XEDAR, the first ligand is EDA-A2; or alternatively if the second receptor is XEDAR, the second ligand is EDA-A2.

Optionally, if the first ligand is TRAIL, the first receptor is selected from the group consisting of TRAIL-R1, TRAIL-R2, TRAIL-R3 and TRAIL-R4; or alternatively if the second ligand is TRAIL, the second receptor is selected from the group consisting of TRAIL-R1, TRAIL-R2, TRAIL-R3 and TRAIL-R4.

Optionally, if the first ligand is TRANCE/RANKL, the first receptor is selected from the group consisting of OPG and RANK; or alternatively if the second ligand is TRANCE/RANKL, the second receptor is selected from the group consisting of OPG and RANK.

Optionally, if the first receptor is TROY, the first ligand is TROY ligand; or alternatively if the second receptor is TROY, the second ligand is TROY ligand.

Optionally, if the first receptor is Fas, the first ligand is FasL; or alternatively if the second receptor is Fas, the second ligand is FasL.

Optionally, if the first receptor is GITR, the first ligand is GITL; or alternatively if the second receptor is GITR, the second ligand is GITL.

Optionally, if the first ligand is LIGHT, the first receptor is selected from the group consisting of DcR3 and HVEM; or alternatively if the second ligand is LIGHT, the second receptor is selected from the group consisting of DcR3 and HVEM.

Optionally, if the first receptor is DR3, the first ligand is TL1A/VEGI; or alternatively if the second receptor is DR3, the second ligand is TL1A/VEGI.

Optionally, if the first receptor is Fn14, the first ligand is TWEAK; or alternatively if the second receptor is Fn14, the second ligand is TWEAK.

Optionally, if the first receptor is TNFR1, the first ligand is TNF-alpha; wherein if the second receptor is TNFR1, the second ligand is TNF-alpha.

Optionally, if the first receptor is TNFR2, the first ligand is TNF-beta; or alternatively if the second receptor is TNFR2, the second ligand is TNF-beta.

Optionally, if the first receptor is Lymphotoxin beta R, the first ligand is selected from the group consisting of LIGHT, lymphotoxin alpha (LTA), and lymphotoxin beta (LTB); or alternatively if the second receptor is Lymphotoxin beta R, the second ligand is selected from the group consisting of LIGHT, lymphotoxin alpha (LTA), and lymphotoxin beta (LTB).

Optionally, if the first receptor is OX40R, the first ligand is OX40L; or alternatively if the second receptor is OX40R, the second ligand is OX40L.

Optionally, if the first receptor is NGFR, the first ligand is selected from the group consisting of NGF and NTF4; or alternatively if the second receptor is NGFR, the second ligand is selected from the group consisting of NGF and NTF4.

Optionally, if the first receptor is DR6, the first ligand is APP; or alternatively if the second receptor is DR6, the second ligand is APP.

Optionally, if the first receptor is RELT, the first ligand is RELT ligand; or alternatively if the second receptor is RELT, the second ligand is RELT ligand.

According to at least some embodiments, there is provided a stabilized composition comprising a fusion protein and a ligand A in a ratio sufficient to increase therapeutic efficacy of the fusion protein in a subject, the fusion protein comprising an extracellular domain (ECD) of a receptor B and an ECD of a ligand C, wherein the ligand C is capable of binding to a receptor D, wherein the ligand A is capable of binding to the receptor B, wherein the ligands A and C, and the receptors B and D, are TNF-family members, and wherein the ligands A and C are different, and the receptors B and D are different.

Optionally, increasing therapeutic efficacy comprises increasing half-life of the fusion protein in the subject.

According to at least some embodiments, there is provided a stabilized composition comprising a fusion protein and a ligand A in a ratio sufficient to increase migration time of the fusion protein in a native PAGE gel, the fusion protein comprising an extracellular domain (ECD) of a receptor B and a ligand C, wherein the ligand C is capable of binding to a receptor D, wherein the ligand A is capable of binding to the receptor B, wherein the ligands A and C, and the receptors B and D, are TNF-family members, and wherein the ligands A and C are different, and the receptors B and D are different.

According to at least some embodiments, there is provided a method of treating a disease in a subject, comprising: determining a level of a ligand A capable of binding to a TNF-family receptor B in the subject; if the level of the ligand A is above a baseline level, administering a fusion protein to the subject to treat the disease, the fusion protein comprising an extracellular domain (ECD) of the receptor B, wherein the fusion protein further comprises a TNF-family ligand C, wherein the ligand C is not capable of binding to the receptor B but wherein the ligand C is capable of binding to a TNF-family receptor D, wherein the disease is treatable by activating the receptor D.

Optionally the method further comprises administering the composition of any of claims 32-34.

Optionally the method further comprises detecting a level of a TNF-family receptor D, to which the ligand C is capable of binding, in the subject; and administering the fusion protein only after detecting the level of the receptor D in the subject.

Optionally if the receptor B is 4-1BB, the ligand A is 4-1BBL; or wherein if the receptor D is 4-1BB, the ligand C is 4-1BBL.

Optionally if the receptor B is BCMA, the ligand A is APRIL or BAFF; or wherein if the receptor D is BCMA, the ligand C is APRIL or BAFF.

Optionally if the receptor B is CD27, the ligand A is CD27L; or wherein if the receptor D is CD27, the ligand C is CD27L.

Optionally if the receptor B is CD30, the ligand A is CD30L; or wherein if the receptor D is CD30, the ligand C is CD30L.

Optionally if the receptor B is CD40, the ligand A is CD40L; or wherein if the receptor D is CD40, the ligand C is CD40L.

Optionally if the receptor B is EDAR, the ligand A is EDA-A1; or wherein if the receptor D is EDAR, the ligand C is EDA-A1.

Optionally if the receptor B is XEDAR, the ligand A is EDA-A2; or wherein if the receptor D is XEDAR, the ligand C is EDA-A2.

Optionally if the ligand A is TRAIL, the receptor B is selected from the group consisting of TRAIL-R1, TRAIL-R2, TRAIL-R3 and TRAIL-R4; or wherein if the ligand C is TRAIL, the receptor D is selected from the group consisting of TRAIL-R1, TRAIL-R2, TRAIL-R3 and TRAIL-R4.

Optionally if the ligand A is TRANCE/RANKL, the receptor B is selected from the group consisting of OPG and RANK; or wherein if the ligand C is TRANCE/RANKL, the receptor D is selected from the group consisting of OPG and RANK.

Optionally if the receptor B is TROY, the ligand A is TROY ligand; or wherein if the receptor D is TROY, the ligand C is TROY ligand.

Optionally if the receptor B is Fas, the ligand A is FasL; or wherein if the receptor D is Fas, the ligand C is FasL.

Optionally if the receptor B is GITR, the ligand A is GITL; or wherein if the receptor D is GITR, the ligand C is GITL.

Optionally if the ligand A is LIGHT, the receptor B is selected from the group consisting of DcR3 and HVEM; or wherein if the ligand C is LIGHT, the receptor D is selected from the group consisting of DcR3 and HVEM.

Optionally if the receptor B is DR3, the ligand A is TL1A/VEGI; or wherein if the receptor D is DR3, the ligand C is TL1A/VEGI.

Optionally if the receptor B is Fn14, the ligand A is TWEAK; or wherein if the receptor D is Fn14, the ligand C is TWEAK.

Optionally if the receptor B is TNFR1, the ligand A is TNF-alpha; wherein if the receptor D is TNFR1, the ligand C is TNF-alpha.

Optionally if the receptor B is TNFR2, the ligand A is TNF-beta; or wherein if the receptor D is TNFR2, the ligand C is TNF-beta.

Optionally, if the first receptor is Lymphotoxin beta R, the first ligand is selected from the group consisting of LIGHT, lymphotoxin alpha (LTA), and lymphotoxin beta (LTB); or alternatively if the second receptor is Lymphotoxin beta R, the second ligand is selected from the group consisting of LIGHT, lymphotoxin alpha (LTA), and lymphotoxin beta (LTB).

Optionally if the receptor B is OX40R, the ligand A is OX40L; or wherein if the receptor D is OX40R, the ligand C is OX40L.

Optionally if the receptor B is NGFR, the ligand A is selected from the group consisting of NGF and NTF4; or wherein if the receptor D is NGFR, the ligand C is selected from the group consisting of NGF and NTF4.

Optionally if the receptor B is DR6, the ligand A is APP; or wherein if the receptor D is DR6, the ligand C is APP.

Optionally if the receptor B is RELT, the ligand A is RELT ligand; or wherein if the receptor D is RELT, the ligand C is RELT ligand.

Optionally a receptor D and a ligand C are selected from TNF-family members such that the receptor D is different from the receptor B and the ligand C is different from the ligand A.

Optionally any of the above compositions or methods may be adapted for treatment of cancer, wherein the cancer is selected from the group consisting of breast, colorectal, hematological, pancreatic, soft tissue sarcoma, cervical, brain, cerebrospinal, bladder, liver, skin and lung cancers.

Optionally, according to at least some embodiments, the cancer is selected from the group consisting of breast cancer, cervical cancer, ovary cancer, endometrial cancer, bladder cancer, lung cancer, pancreatic cancer, colon cancer, prostate cancer, leukemia, B-cell lymphoma, Burkitt's lymphoma, multiple myeloma, Hodgkin's lymphoma, Non-Hodgkin's lymphoma, thyroid cancer, thyroid follicular cancer, myelodysplastic syndrome (MDS), fibrosarcomas and rhabdomyosarcomas, melanoma, uveal melanoma, teratocarcinoma, neuroblastoma, glioma, glioblastoma, benign tumor of the skin, keratoacanthomas, renal cancer, anaplastic large-cell lymphoma, esophageal squamous cells carcinoma, follicular dendritic cell carcinoma, intestinal cancer, muscle-invasive cancer, seminal vesicle tumor, epidermal carcinoma, spleen cancer, bladder cancer, head and neck cancer, stomach cancer, liver cancer, bone cancer, brain cancer, cancer of the retina, biliary cancer, salivary gland cancer, cancer of uterus, cancer of testicles, cancer of connective tissue, prostatic hypertrophy, myelodysplasia, Waldenstrom's macroglobinaemia, nasopharyngeal, neuroendocrine cancer, mesothelioma, angiosarcoma, Kaposi's sarcoma, carcinoid, oesophagogastric, fallopian tube cancer, peritoneal cancer, papillary serous mullerian cancer, malignant ascites, gastrointestinal stromal tumor (GIST), Li-Fraumeni syndrome and Von Hippel-Lindau syndrome (VHL), and wherein the cancer is non-metastatic, invasive or metastatic.

Optionally the leukemia is acute lymphocytic leukemia, chronic lymphocytic leukemia, or myeloid leukemia; and/or the liver cancer is hepatocellular carcinoma; and/or the intestinal cancer is small bowel cancer.

Optionally the myeloid leukemia is acute myelogenous leukemia (AML) or chronic myelogenous leukemia.

Optionally any of the above compositions or methods may be adapted for treatment of an inflammatory or immune related condition, for example for treatment of an autoimmune disease.

Optionally the autoimmune disease is selected from the group consisting of rheumatoid arthritis, hematological autoimmune diseases, multiple sclerosis, autoimmune diabetes, SLE (systemic lupus erythematosus), ANCA-associated vasculitis (ANCA stands for anti-neutrophil cytoplasmic antibody) and autoimmune thyroiditis.

According to at least some embodiments, optionally the autoimmune disease is selected from the group consisting of multiple sclerosis, including relapsing-remiting multiple sclerosis, primary progressive multiple sclerosis, and secondary progressive multiple sclerosis; rheumatoid arthritis; psoriatic arthritis, systemic lupus erythematosus, (SLE); lupus nephritis; ulcerative colitis; Crohn's disease; benign lymphocytic angiitis, thrombocytopenic purpura, idiopathic thrombocytopenia, idiopathic autoimmune hemolytic anemia, pure red cell aplasia, Sjogren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, cryoglobulinemic vasculitis, ANCA-associated vasculitis, antiphospholipid syndrome, myasthenia gravis, autoimmune haemolytic anaemia, Guillian-Barre syndrome, chronic immune polyneuropathy, autoimmune thyroiditis, insulin dependent diabetes mellitus, type I diabetes, Addison's disease, membranous glomerulonephropathy, Focal Segmental glomerulonephritis, mesangiocapillary glomerulonephritis, post infection glomerulonephritis, Fibrillary, immunotactoid glomerulonephritis, anti-GBM (including but not limited to Goodpasture's disease), autoimmune gastritis, autoimmune atrophic gastritis, pernicious anaemia, pemphigus, pemphigus vulgarus, cirrhosis, primary biliary cirrhosis, dermatomyositis, polymyositis, fibromyositis, myogelosis, celiac disease, immunoglobulin A nephropathy, C3 nephropathy, Immune-complex glomerulonephritis, Henoch-Schonlein purpura, Evans syndrome, atopic dermatitis, psoriasis, psoriasis arthritis, Graves' disease, Graves' ophthalmopathy, scleroderma, systemic scleroderma, progressive systemic scleroderma, asthma, allergy, primary biliary cirrhosis, Hashimoto's thyroiditis, primary myxedema, sympathetic ophthalmia, autoimmune uveitis, hepatitis, chronic action hepatitis, collagen diseases, ankylosing spondylitis, periarthritis humeroscapularis, panarteritis nodosa, chondrocalcinosis, Wegener's granulomatosis, microscopic polyangiitis, chronic urticaria, bullous skin disorders, pemphigoid, atopic eczema, Devic's disease, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, pancreatitis, myocarditis, vasculitis, gastritis, gouty arthritis, and inflammatory skin disorders, selected from the group consisting of psoriasis, atopic dermatitis, eczema, rosacea, urticaria, and acne, normocomplementemic urticarial vasculitis, pericarditis, myositis, anti-synthetase syndrome, scleritis, macrophage activation syndrome, Bechet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, autoimmune retinopathy, age-related macular degeneration, atherosclerosis, chronic prostatitis and TNF receptor-associated periodic syndrome (TRAPS).

Other diseases are: Seborrheic keratosis, Inflammatory bowel disease (IBD), Lupus Nephritis, Multiple sclerosis, Parkinson's disease, Amyotrophic lateral sclerosis (ALS), Atopic dermatitis, Psoriasis vulgaris, Urticarial vasculitis, Myocardial infarction, Proliferative diabetic retinopathy or Acute ischemic stroke

The below table, Table 2, relates to some exemplary, non-limiting relationships between TNF-superfamily ligands/receptors with various diseases.

TABLE 2
List of TNF superfamily ligands/receptors with various diseases

| Ligand | Receptor | Autoimmunity | Cancer |
|---|---|---|---|
| 4-1BBL | 4-1BB | wegener's | carcinoma |
| | | granulomatosis | solid tumors |
| | | multiple sclerosis | melanoma |
| 4-1BBL | 4-1BB | rheumatoid arthritis | dendritic cell tumor |
| | | graves disease | non-Hodgkin's |
| | | | lymphoma |
| APRIL | TACI | systemic lupus | brain glioblastoma |
| | BCMA | erythematosus | multiforme |
| | | rheumatoid arthritis | glioblastoma |
| | | multiple sclerosis | multiforme |
| | | | leukemia |
| | | | lymphocytic chronic |
| | | | hodgkin disease |
| | | | hematologic |
| | | | malignancies |
| | | | lymphoma |
| | | | non-Hodgkin's |
| | | | lymphoma |
| | | | multiple myeloma |
| | | | Waldenström's |
| | | | macroglobulinaemia |
| APRIL | TACI | | myeloma |
| BAFF | | | leukemia |
| | | | lymphocytic chronic |
| | | | lymphoma non- |
| | | | hodgkins |
| BAFF | BCMA | lupus | non-hodgkin |
| APRIL | | erythematosus | lymphoma |
| | | systemic | Multiple myeloma |
| | | rheumatoid arthritis | Myeloma |
| | | | leukemia |
| | | | lymphocytic chronic |
| | | | lymphoma t-cell |
| BAFF | TACI | Systemic lupus | chronic lymphocytic |
| | BCMA | erythematosus | leukemia |
| | BAFF-R | pediatric systemic | familial chronic |
| | | lupus | lymphocytic |
| | | erythematosus | leukemia |
| | | systemic lupus | Lymphoma |
| | | erythematosus | non-Hodgkin's |
| | | myasthenia gravis | lymphoma |
| | | pemphigus vulgaris | Multiple myeloma |
| | | Rheumatoid | Waldenström's |
| | | arthritis | macroglobulinaemia |
| | | Sjogren's syndrome | |
| | | multiple sclerosis | |
| BAFF | BAFFR | rheumatic disease | b-cell lymphomas |
| | | systemic lupus | central nervous |
| | | erythematosus | system lymphoma |
| | | | non-hodgkin |
| | | | lymphom |
| | | | multiple myeloma |
| | | | lymphoma follicular |
| CD27L | CD27 | subacute cutaneous | mantle cell |
| | | lupus | lymphoma |
| | | erythematosus | non-hodgkin |
| | | lupus | lymphoma |
| | | erythematosus | stomach cancer |
| | | systemic | leukemia b-cell |
| | | | lymphocytic |
| | | | leukemia chronic b- |
| | | | cell |
| | | | carcinoma renal cell |
| | | | glioblastoma |
| | | | lymphoma |
| | | | renal |
| | | | melanoma |
| CD27L | CD27 | transverse myelitis | lymphoma small |
| | | | lymphocytic |
| | | | leukemia b-cell |
| | | | leukemia |
| | | | lymphocytic chronic |
| | | | lymphoma |
| | | | lymphocytic |
| | | | leukemia chronic b- |
| | | | cell |
| | | | lymphoma follicular |
| | | | plasmacytoma |
| CD30L | CD30 | takayasu's arteritis | hodgkin's lymphoma |
| | | | anaplastic large cell |
| | | | lymphoma |
| | | | lymphocytic |
| | | | leukemia chronic b- |
| | | | cell |
| | | | leukemia hairy cell |
| | | | lymphoproliferative |
| | | | disorders |
| CD30L | CD30 | | hodgkin's lymphoma |
| | | | composite lymphoma |
| | | | reticulosarcoma |
| | | | malignant |
| | | | histiocytosis |
| | | | lymphoepithelioma- |
| | | | like carcinoma |
| | | | primary mediastinal |
| | | | large b-cell |
| | | | lymphoma |
| | | | intravascular large b- |
| | | | cell lymphoma |
| | | | Lymphoma |
| | | | anaplastic large cell |
| | | | lymphoma |
| | | | lymphoma large cell |
| | | | lymphoma |
| | | | pleomorphic |
| | | | lymphoma t-cell |
| | | | peripheral |
| | | | lymphoma t-cell |
| | | | lymphoproliferative |
| | | | disorders |
| CD40L | CD40 | Lupus | Lymphoma |
| | | erythematosus | Leukemia |
| | | Diabetes mellitus | |
| | | Rheumatoid | |
| | | arthritis | |
| | | Psoriatic arthritis | |
| | | psoriasis | |
| CD40L | CD40 | autoimmune | plasma cell leukemia |
| | | thrombocytopenic | monocytic leukemia |
| | | purpura | cervical squamous |
| | | graves' disease | cell carcinoma |
| | | Multiple sclerosis | acute monocytic |
| | | Rheumatoid | leukemia |
| | | arthritis | chronic lymphocytic |
| | | | leukemia |
| | | | Lymphoma |
| | | | Leukemia |
| | | | Multiple myeloma |
| | | | lymphocytic |
| | | | leukemia chronic b- |
| | | | cell |
| | | | Hodgkin's lymphoma |
| | | | non-Hodgkin's |
| | | | lymphoma |
| | | | follicular lymphoma |
| | | | melanoma |
| | | | pancreatic cancer |
| EDA isoform A1 | EDA1R2; EDAR | | |
| EDA isoform A2 | XEDAR2 | | |
| TRAIL | TRAILR1 | | colon cancer |
| | TRAILR2 | | malignant glioma |
| | TRAILR3 | | glioma |
| | TRAILR4 | | prostate cancer |
| | OPG | | hepatocellular |
| | | | carcinoma |
| | | | glioblastoma |
| | | | adult |
| | | | medulloblastoma |
| | | | thoracic cancer |
| | | | prostate cancer |
| | | | pancreatic cancer |
| | | | medulloblastoma |
| TRAIL | TRAIL-R1 | | lung cancer |
| | | | ameloblastoma |
| | | | prostate cancer |
| | | | colon cancer |
| | | | leukemia |
| | | | lymphocytic chronic |
| | | | myeloma |
| | | | malignant glioma |
| | | | ovarian cancer |
| TRAIL | TRAIL-R2 | | squamous cell |
| | | | carcinoma of the head |
| | | | and neck |
| | | | pancreatic cancer |
| | | | oral cavity cancer |
| | | | colon cancer |
| | | | malignant glioma |
| | | | glioma |
| | | | cancer lung |
| | | | head cancer |
| | | | carcinoma renal cell |
| TRAIL | TRAIL-R3 | | pancreatic cancer |
| | | | myeloma |
| | | | ovarian cancer |
| | | | malignant |
| | | | mesothelioma |
| | | | prostate cancer |
| | | | glioma |
| | | | adenoma |
| | | | colon cancer |
| TRAIL | TRAIL-R4 | | prostate cancer |
| | | | colon carcinoma |
| | | | ovarian cancer |
| | | | glioma |
| | | | osteosarcoma |
| | | | colon cancer |
| RANKL | OPG | Arthritis | bone cancer |
| TRAIL | | | optic nerve glioma |
| | | | Multiple myeloma |
| | | | bone metastasis |
| RANKL | OPG | Rheumatoid | Multiple myeloma |
| | RANK | arthritis | Giant cell tumor |
| | | | prostate cancer |
| | | | ameloblastoma |
| | | | myeloma |
| RANKL | RANK | | osteosarcoma |
| Unknown Lig. | TROY | | colorectal cancer |
| | | | intestinal tumors |
| FASL | FAS | Fasl related | lung cancer |
| | DcR3 | autoimmune | non-small cell lung |
| | | lymphoproliferative | carcinoma |
| | | syndrome | epstein-barr virus- |
| | | autoimmune | associated gastric |
| | | lymphoproliferative | carcinoma |
| | | syndrome | ras-associated |
| | | hashimotos | autoimmune |
| | | thyroiditis | leukoproliferative |
| | | | disease |
| | | | malignant glioma |
| | | | leukemia |
| | | | lymphocytic large |
| | | | granular |
| | | | colorectal carcinoma |
| | | | gastric cancer |
| | | | cervical carcinoma |
| | | | pancreatic cancer |
| FASL | FAS | autoimmune | squamous cell |
| | | lymphoproliferative | carcinoma |
| | | syndrome, type ia | ovarian serous |
| | | dianzani | cystadenocarcinoma |
| | | autoimmune | thyroid lymphoma |
| | | lymphoproliferative | malignant glioma |
| | | disease | pediatric |
| | | hashimotos | osteosarcoma |
| | | thyroiditis | osteosarcoma |
| GITRL | GITR | | |
| GITRL | GITR | rheumatoid arthritis | B16 melanoma |
| | | wegener's | |
| | | granulomatosis | |
| LIGHT | HVEM | rheumatoid arthritis | |
| | LTBR | inflammatory | |
| | DcR3 | bowel diseases | |
| | | colitis | |
| BTLA | HVEM | rheumatoid arthritis | follicular lymphoma |
| LIGHT | DcR3 | autoimmune | oral cavity cancer |
| homotrimeric LTA | | hepatitis | |
| LIGHT | | rheumatoid arthritis | colon |
| | | | adenocarcinoma |
| TL1A | | | malignant glioma |
| FASL | | | pancreatic cancer |
| | | | esophageal squamous |
| | | | cell carcinoma |
| | | | pancreatic carcinoma |
| | | | colon |
| | | | adenocarcinoma |
| | | | hepatocellular |
| | | | carcinoma |
| | | | gastric carcinoma |
| TL1A | DR3 | inflammatory | |
| | DcR3 | bowel disease | |
| | | irritable bowel | |
| | | syndrome | |
| | | Crohn's disease | |
| | | Ulcerative colitis | |
| TWEAK* | DR3 | type 1 diabetes | |
| | | mellitus | |
| | | Rheumatoid | |
| | | arthritis | |
| TWEAK | FN14 | type 1 diabetes | Glioblastoma |
| | DR3 | mellitus | multiforme |
| | | rheumatoid arthritis | |
| | | systemic lupus | |
| | | erythematosus | |
| | | multiple sclerosis | |
| TWEAK | FN14 | multiple sclerosis | glioblastoma |
| | | rheumatoid arthritis | multiforme |
| | | lupus nephritis | glioblastoma |
| | | | breast cancer |
| TNF-alpha | TNFR1 | Psoriatic arthritis | |
| | TNFBR | Rheumatoid | |
| | | arthritis | |
| | | Crohn's disease | |
| | | Psoriasis | |
| | | Ankylosing | |
| | | spondylitis | |
| | | Multiple sclerosis | |
| | | Diabetes mellitus | |
| | | Ulcerative colitis | |
| TNF-alpha | TNF-R1 | multiple sclerosis | |
| LTA | | Arthritis; psoriatic | |
| | | arthritis | |
| | | Inflammatory | |
| | | bowel disease | |
| | | pediatric systemic | |
| | | lupus | |
| | | systemic lupus | |
| | | erythematosus | |
| | | Rheumatoid | |
| | | arthritis | |
| | | psoriatic arthritis | |
| | | psoriasis | |
| | | ankylosing | |
| TNF-alpha | TNFR2 | rheumatoid arthritis | sarcoma |
| LTA | | lupus | solid tumour |
| | | erythematosus | |
| | | systemic | |
| | | arthritis psoriatic | |
| | | Ankylosing | |
| | | spondylitis | |
| | | guillain-barre | |
| | | syndrome | |
| | | spondylitis | |
| | | behcet's disease | |
| | | rheumatoid arthritis | |
| | | Psoriasis | |
| | | Psoriatic arthritis | |
| | | Crohn's disease | |
| | | ulcerative colitis | |
| LTA | TNFRSF1A/TNFR1 | Psoriatic arthritis | non-hodgkin |
| | TNFRSF1B/TNFBR | arthritis | lymphoma |
| | HVEM | multiple sclerosis | T cell Leukemia |
| | LTBR | systemic sclerosis | |
| | | polymyositis | |
| LTB | LTBR | Rheumatoid | non-hodgkin |
| | | arthritis, | lymphoma |
| | | Sjögren's | Leukemia |
| | | syndrome | |
| LIGHT | LTBR | colitis | |
| | | inflammatory | |
| | | bowel diseases | |
| OX40L | OX40 | Systemic lupus | leukemia t-cell |
| | | erythematosus | |
| | | takayasu's arteriti | |
| | | vasculitis | |
| OX40L | OX40 | rheumatoid arthritis | leukemia t-cell |
| | | myasthenia gravis | ductal carcinoma in |
| | | wegener's | situ |
| | | granulomatosis | thymoma |
| | | graves' disease | solid tumor |
| | | | prostate |
| NGF | NTRK1 | Diabetes mellitus | ependymoblastoma |
| | NGFR | | ependymoblastoma |
| | | | askin's tumor |
| | | | Neuroblastoma |
| | | | Phaeochromocytoma |
| BDNF | NTRK2 | Multiple sclerosis | Neuroblastoma |
| NTF3 | NTRK1 | | ganglioneuroma |
| | | | medulloblastoma |
| NTF4 | NGFR | relapsing-remitting | tuberous sclerosis |
| | | multiple sclerosis | complex |
| | | | epithelial tumor |
| NGF | NTRK1 | | Thyroid papillary |
| NTF3 | | | carcinoma |
| | | | familial medullary |
| | | | thyroid carcinoma |
| | | | ntrk1-related familial |
| | | | medullary thyroid |
| | | | carcinoma |
| | | | thyroid medullary |
| | | | carcinoma |
| | | | adrenal |
| | | | neuroblastoma |
| | | | askin's tumor |
| | | | follicular thyroid |
| | | | carcinoma |
| | | | neuroblastoma |
| | | | granular cell tumor |
| | | | ganglioneuroma |
| | | | stage neuroblastoma |
| | | | pheochromocytoma |
| | | | ganglioneuroblastoma |
| | | | medulloblastoma |
| | | | pancreatic cancer |
| BDNF | NTRK2 | | pilocytic astrocytoma |
| NTF3 | | | neuroblastoma |
| NTF4 | | | medulloblastoma |
| | | | ganglioneuroma |
| | | | ganglioglioma |
| | | | myeloma |
| NT3 | NTRK3 | | congenital |
| | | | fibrosarcoma |
| | | | fibrosarcoma. |
| | | | congenital |
| | | | mesoblastic |
| | | | nephroma |
| | | | medulloblastoma |
| | | | mesoblastic |
| | | | nephroma |
| | | | polymorphous low- |
| | | | grade |
| | | | adenocarcinoma |
| | | | gastrointestinal |
| | | | stromal tumor |
| | | | desmoplastic |
| | | | medulloblastoma |
| | | | leukemia mast cell |
| | | | gastrointestinal |
| | | | stromal tumor |
| | | | mastocytoma |
| | | | sarcoma spindle cell |
| | | | pediatric solid tumor |
| NGF | NGFR | | basal cell carcinoma |
| BDNF | | | malignant peripheral |
| NT3 | | | nerve sheath tumor |
| NT4 | | | epithelioid malignant |
| | | | peripheral nerve |
| | | | sheath |
| | | | pediatric |
| | | | ependymoma |
| | | | medulloblastoma |
| | | | thymic carcinoma |
| | | | neuroblastoma |
| | | | mucosal melanoma |
| | | | Melanoma |
| N-APP | TNFRSF21 | | Lung cancer |
| | | | Carcinoma |

Please note that additional diseases associated with TWEAK are described herein in the application.
Italics font of the ligand/receptor stands for the ECD in the fusion protein (e.g. B or C) whereas regular font stands for the molecule that binds to it (e.g. A or D respectively).

According to at least some embodiments, there is provided a composition comprising Fn14-TRAIL fusion protein and TWEAK in a pharmaceutically effective amount to induce apoptosis.

Optionally an amount of the Fn14-TRAIL fusion protein is in a range of from 0.001 mg to 20 mg of the Fn14-TRAIL fusion protein per kg body weight of a subject receiving the Fn14-TRAIL fusion protein.

Optionally the amount is from 0.1 mg to 5 mg per kg body weight.

Optionally an amount of the TWEAK protein is in a range of from 0.001 mg to 20 mg of the TWEAK protein per kg body weight of a subject receiving the TWEAK protein.

Optionally the amount is from 0.1 mg to 5 mg.

Optionally the TWEAK protein further comprises a half-life extending moiety, with the proviso that the half-life extending moiety does not affect formation of a trimer comprising the TWEAK protein.

Optionally the half-life extending moiety comprises polyethylene glycol (PEG), monomethoxy PEG (mPEG), an XTEN molecule, an rPEG molecule, an adnectin, a serum albumin, human serum albumin, acyl group or heterologous peptide.

Optionally the Fn14-TRAIL fusion protein further comprises a half-life extending moiety.

Optionally the half-life extending moiety comprises polyethylene glycol (PEG), monomethoxy PEG (mPEG), or an XTEN molecule.

Optionally the addition of the half-life extending moiety increases the in vivo half-life of the fusion protein and/or the TWEAK by at least about 2-fold, at least about 3-fold, at least about 4-fold, at least about 5-fold, at least about 10-fold, or more, as compared to the identical molecule without such the half-life extending moiety.

Optionally the Fn14-TRAIL fusion protein and the TWEAK protein are in a ratio of from 50:1 to 1:50.

Optionally the ratio is from 20:1 to 1:20.

Optionally the ratio is from 10:1 to 1:10.

Optionally the ratio is from 5:1 to 1:5.

Optionally the ratio is from 2:1 to 1:2.

According to at least some embodiments, there is provided a composition comprising Fn14-TRAIL fusion protein and TWEAK protein in a ratio in a range of from 50:1 to 1:50.

Optionally the ratio is from 20:1 to 1:20.

Optionally the ratio is from 10:1 to 1:10.

Optionally the ratio is from 5:1 to 1:5.

Optionally the ratio is from 2:1 to 1:2.

According to at least some embodiments there is provided a stabilized composition comprising Fn14-TRAIL fusion protein and TWEAK protein in a ratio sufficient to increase half-life of Fn14-TRAIL fusion protein in a subject.

Optionally the Fn14-TRAIL fusion protein and the TWEAK protein are in a ratio of from 50:1 to 1:50.

Optionally the ratio is from 20:1 to 1:20.

Optionally the ratio is from 10:1 to 1:10.

Optionally the ratio is from 5:1 to 1:5.

Optionally the ratio is from 2:1 to 1:2.

According to at least some embodiments, there is provided a method of treating cancer in a subject, comprising administering a composition comprising the Fn14-TRAIL fusion protein according to the embodiments of the invention to the subject, wherein the cancer is selected from the group consisting of any cancer that can be treated by activating the TRAIL pathway and wherein TWEAK can be detected or its level is elevated in a diseased organ, tissue or in their environment or in the blood, Cerebrospinal Fluid (CSF), synovial fluid, SALIVA, or urine or any other body fluids or excretions of the subject inflicted with the cancer, in comparison to healthy subject.

According to some embodiments, the cancer is testis cancer, urothelial cancer, Hodgkin lymphoma, squamous cell carcinoma, keratinocyte carcinoma or osteosarcoma.

According to at least some embodiments, there is provided a method of treating an autoimmune disease in a subject, comprising administering the composition of any of the above claims to the subject, wherein the autoimmune disease is selected from the group consisting of any autoimmune disease that can be treated by activating the TRAIL pathway.

According to at least some embodiments, there is provided a method of treating a disease treatable by activating TRAIL receptors in a subject, comprising testing cells related to the disease for the presence of TWEAK; if TWEAK is detected, administering Fn14-TRAIL to the subject.

Optionally the TWEAK is detected at a local disease site.

Optionally the TWEAK is present at a sufficiently high level at the local disease site.

According to at least some embodiments of the present invention, the component 1 protein is a type-I membrane protein, while the component 2 protein is a type-II membrane protein. Optionally and preferably for such embodiments, the bi-component protein comprises the extracellular domain of the type-I membrane protein and the extracellular domain of the type-II membrane protein. Optionally and more preferably, the bi-component protein has an N-terminal side which is the extracellular domain of the type-I membrane protein and a C-terminal side which is composed of the extracellular domain of the type-II membrane protein. Optionally and more preferably, the type-I membrane protein is a TNF-superfamily receptor and the type-II membrane protein is a TNF-superfamily ligand. Optionally and more preferably, the TNF-superfamily receptor is designated “B” in the above embodiment and the TNF-superfamily ligand is designated “C” in the above embodiment.

“TWEAK-related condition/s” or “disease/s associated with TWEAK” or “condition/s associated with TWEAK” are interchangeably refer to any conditions that result from aberrant TWEAK function or regulation. The term may also refer to any condition that does not directly result from aberrant TWEAK function or regulation, but rather arises out of some other mechanism wherein disrupting, increasing or otherwise altering TWEAK activity will have a detectable outcome on the condition. TWEAK-related conditions can be either inflammatory or non-inflammatory in nature, and include, but are not limited to, the conditions and diseases specifically disclosed herein, including without limitation cancer and autoimmune diseases as well as other diseases as described herein.

As used herein, the term “fusion proteins” refers to chimeric proteins comprising amino acid sequences of two or more different proteins. Typically, fusion proteins result from in vitro recombinatory techniques well known in the art.

As used herein, “biologically active or immunologically active” refers to fusion proteins according to the present invention having a similar structural function (but not necessarily to the same degree), and/or similar regulatory function (but not necessarily to the same degree), and/or similar biochemical function (but not necessarily to the same degree) and/or immunological activity (but not necessarily to the same degree) as the individual wild type proteins which are the building blocks of the fusion proteins of the present invention.

As used herein, a “deletion” is defined as a change in amino acid sequence in which one or more amino acid residues are absent as compared to the wild-type protein.

As used herein an “insertion” or “addition” is a change in an amino acid sequence that has resulted in the addition of one or more amino acid residues as compared to the wild-type protein.

As used herein “substitution” results from the replacement of one or more amino acids by different amino acids, respectively, as compared to the wild-type protein.

As used herein, the term “variant” means any polypeptide having a substitution of, deletion of or addition of one (or more) amino acid from or to the sequence (or any combination of these), including allelic variations, as compared with the wild-type protein, so long as the resultant variant fusion protein retains at least 75%, 80%, 85%, 90%, 95%, 99% or more of the biological or immunologic activity as compared to the wild-type proteins as used in the present invention. Typically, variants of the FN14/TRAIL fusion protein embraced by the present invention will have at least 80% or greater sequence identity or homology, as those terms are understood in the art, to SEQ. ID. NO. 1, more preferably at least 85%, 86%, 87%, 88%, 89%, 90%, 91%, 92%, 93%, 94%, 95%, 96%, 97%, 98%, or even 99% sequence identity to SEQ. ID. NO. 1.

Sequence identity or homology can be determined using standard techniques known in the art, such as the Best Fit sequence program described by Devereux et al., Nucl. Acid Res. 12:387-395 (1984) or the BLASTX program (Altschul et al., J. Mol. Biol. 215, 403-410). The alignment may include the introduction of gaps in the sequences to be aligned. In addition, for sequences which contain either more or fewer amino acids than the proteins disclosed herein, it is understood that the percentage of homology will be determined based on the number of homologous amino acids in relation to the total number of amino acids.

Additionally, while in general it is desirable for variants to show enhanced ability for binding to a given molecule, in some embodiments variants may be designed with slightly reduced activity as compared to other fusion proteins of the invention, for example, in instances in which one would purposefully want to attenuate activity, for example, to diminish neurotoxicity. Moreover, variants or derivatives can be generated that would bind more selectively to one of the TRAIL receptor variants (there are five TRAIL receptors in humans, two activating and three decoy receptors). Furthermore, variants or derivatives can be generated that would have altered multimerization properties. When engineering variants, this could be done for either the entire TRAIL extracellular domain, or for that component of the extracellular domain that is incorporated within the fusion protein itself.

Preferably, variants or derivatives of the fusion proteins of the present invention maintain the hydrophobicity/hydrophilicity of the amino acid sequence. Conservative amino acid substitutions may be made, for example from 1, 2 or 3 to 10, 20 or 30 substitutions provided that the modified sequence retains the ability to act as a fusion protein in accordance with present invention. Amino acid substitutions may include the use of non-naturally occurring analogues, for example to increase blood plasma half-life.

The present invention relates to a method of treating a disease in a subject, wherein the disease is associated with presence or high level of a first TNF-family ligand in comparison to a healthy subject, as measured in a diseased tissue or organ or in the envornment or in the blood, Cerebrospinal Fluid (CSF), synovial fluid, saliva, or urine or any other body fluids or excretions of the subject, the method comprising: administering a fusion protein to the subject to treat the disease, the fusion protein comprising an extracellular domain (ECD) of a first TNF-family receptor, wherein the fusion protein further comprises a second TNF-family ligand, wherein the second TNF-family ligand is not capable of binding to the first receptor but wherein the first TNF-family ligand is capable of binding to the first TNF-family receptor thereby treating the disease in the subject.

The invention relates to a method of treating a disease in a subject, comprising: administering a fusion protein to the subject to treat the disease, the fusion protein comprising an extracellular domain (ECD) of a first TNF-family receptor, wherein the fusion protein further comprises a second TNF-family ligand, wherein the second TNF-family ligand is not capable of binding to the first receptor but wherein the second TNF-family ligand is capable of binding to a second TNF-family receptor, wherein the disease is treatable by activating the second TNF-family receptor.

The invention relates to a method of treating a disease in a subject, comprising: determining a presence or a level of a first TNF-family ligand capable of binding to a first TNF-family receptor in the subject; if said level of said first ligand is above a baseline level, administering a fusion protein to the subject to treat the disease, the fusion protein comprising an extracellular domain (ECD) of said first TNF-family receptor, wherein said fusion protein further comprises a second TNF-family ligand, wherein said second TNF-family ligand is not capable of binding to said first receptor but wherein said second TNF-family ligand is capable of binding to a second TNF-family receptor, wherein said disease is treatable by activating said second TNF-family receptor.

The invention relates to a stabilized composition comprising a fusion protein and a first ligand in a ratio sufficient to increase therapeutic efficacy of the fusion protein in a subject, the fusion protein comprising an extracellular domain (ECD) of a first receptor and a second ligand, wherein the second ligand is capable of binding to a second receptor, wherein the first ligand is capable of binding to the first receptor, wherein the first and second ligands, and the first and second receptors, are TNF-family members, and wherein the first and second ligands are different, and the first and second receptors are different.

The invention relates to a stabilized composition comprising a fusion protein and a first ligand in a ratio sufficient to increase migration time of the fusion protein in a native PAGE gel, the fusion protein comprising an extracellular domain (ECD) of a first receptor and a second ligand, wherein the second ligand is capable of binding to a second receptor, wherein the first ligand is capable of binding to the first receptor, wherein the first and second ligands, and the first and second receptors, are TNF-family members, and wherein the first and second ligands are different, and the first and second receptors are different.

The invention relates to a composition comprising Fn14-TRAIL fusion protein and TWEAK in a pharmaceutically effective amount to induce apoptosis.

The invention relates to a complex comprising a fusion protein and a first ligand, the fusion protein comprising an extracellular domain (ECD) of a first receptor and a second ligand, wherein the second ligand is capable of binding to a second receptor, wherein the first ligand is capable of binding to the first receptor, wherein the first and second ligands, and the first and second receptors, are TNF-family members, and wherein the first and second ligands are different, and the first and second receptors are different.

### BRIEF DESCRIPTION OF THE DRAWINGS

FIGS. 1A-E

Susceptibility of Leukemia Cells to Apoptosis by Fn14-TRAIL Fusion Protein (Fn14-TRAIL) is Increased in the Presence of TWEAK

FIG. 1A. Cells (0.5*10⁶/mL) of four lymphoblastoid lines (Jurkat, J Y, Daudy and Raji) were cultured for 24 h in vitro with 25 ng/mL, 250 ng/mL, or 500 ng/mL of Fn14-TRAIL (“FT”) fusion protein or TRAIL, (FIG. 1B) and tested by flow cytometry for percentage of apoptotic cells over untreated control. FIG. 1C. Jurkat cells were cultured for 24 h in vitro with 250 ng/mL of Fn14-TRAIL, Fn14, TRAIL, or Fn14 and TRAIL in combination before percentage of apoptotic cells over untreated control was tested. FIG. 1D. Percentage of apoptotic cells over untreated control was detected by flow cytometry in four lymphoblastoid cell lines incubated for 24 h in vitro in medium containing 100 ng/mL TWEAK or 100 ng/mL TWEAK in combination with 25 ng/mL, 250 ng/mL or 500 ng/mL Fn14-TRAIL. Bars represent mean±SE of triplicate experiments (FIG. 1A, FIG. 1B, FIG. 1C, FIG. 1D).

FIG. 1E. is a representative density plot analysis of apoptosis induced in Jurkat cells by 24 h incubation with 250 ng/mL Fn14-TRAIL, 250 ng/mL TRAIL, 250 ng/mL Fn14-TRAIL in combination with 100 ng/mL TWEAK, or 250 ng/mL TRAIL in combination with 100 ng/mL TWEAK.

FIGS. 2A-D

Anti-DR5 and Anti-TRAIL Neutralizing Antibodies Interfere with Apoptosis Induction Both by Fn14-TRAIL and Fn14-TRAIL and TWEAK Combination, while Anti-Fn14 and Anti-TWEAK Neutralizing Antibodies Inhibit Only Apoptosis Induced by Fn14-TRAIL and TWEAK

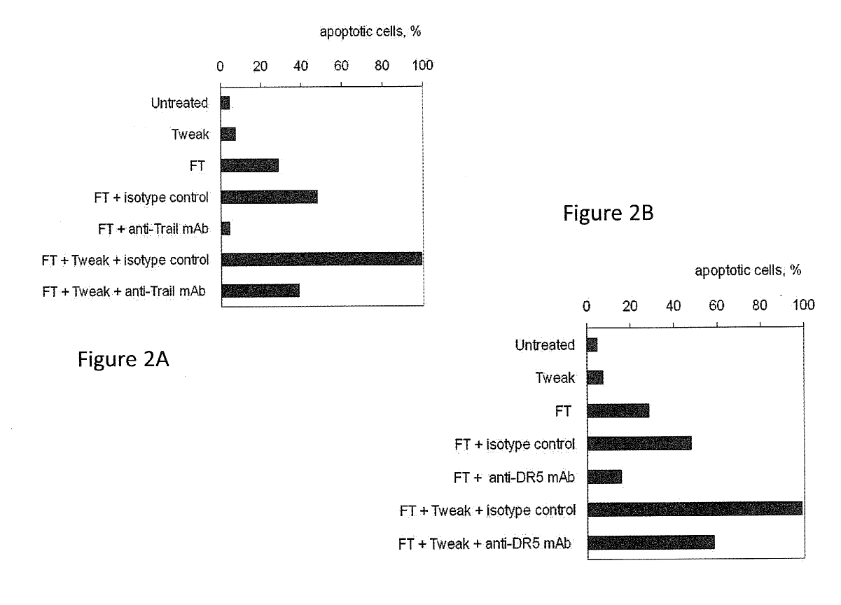

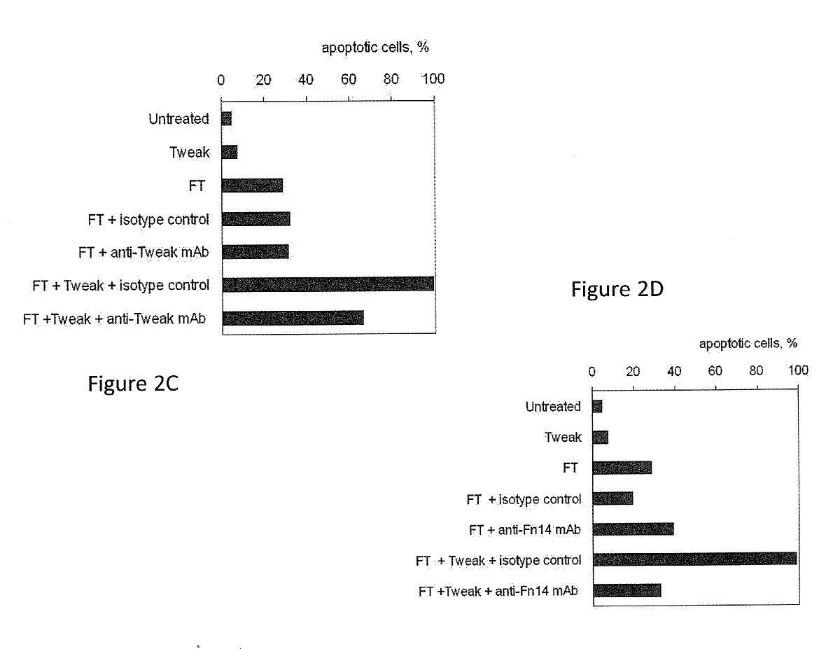

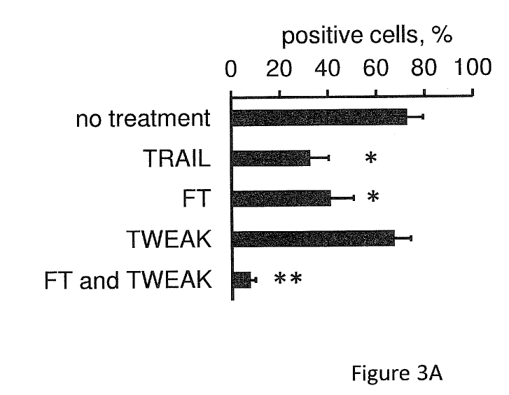

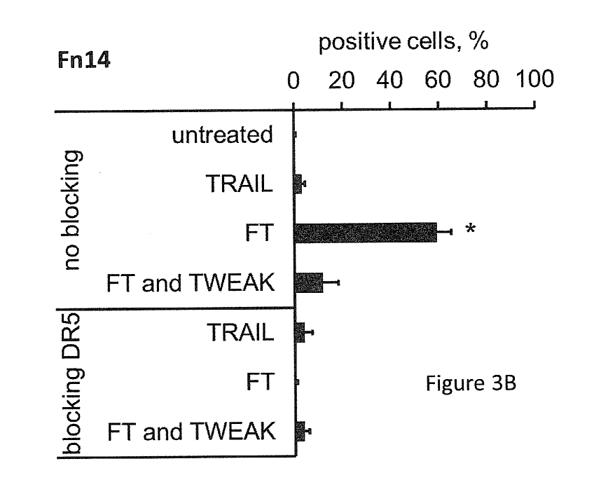

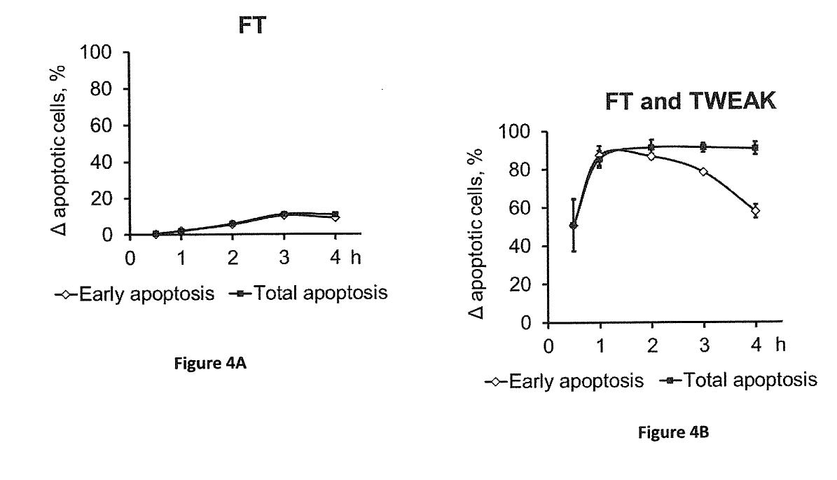

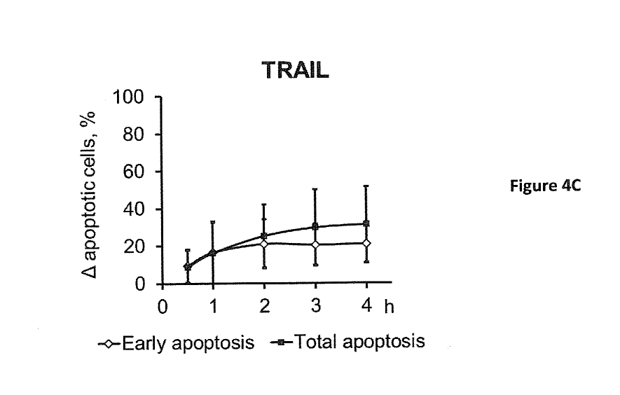

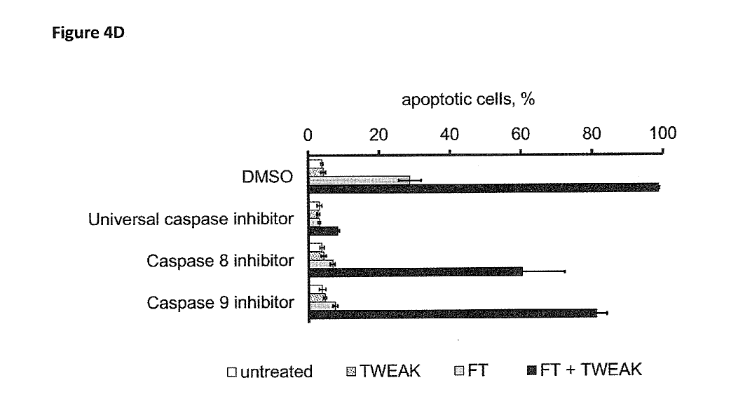

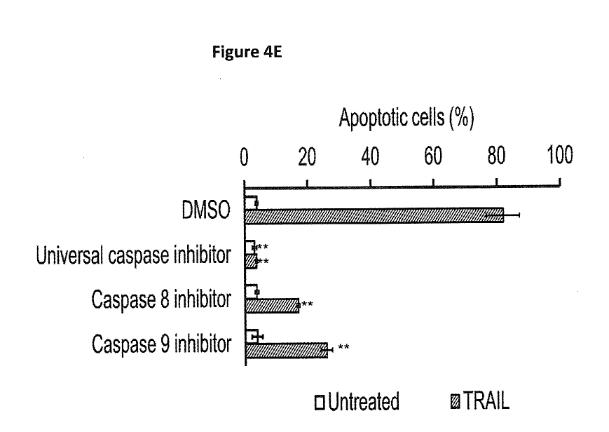

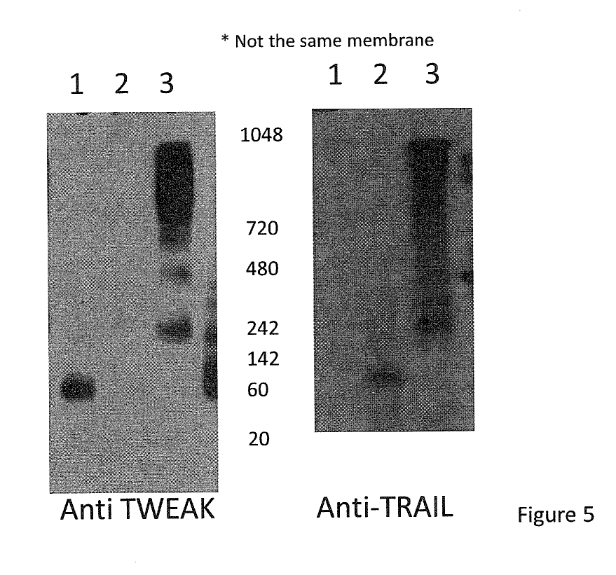

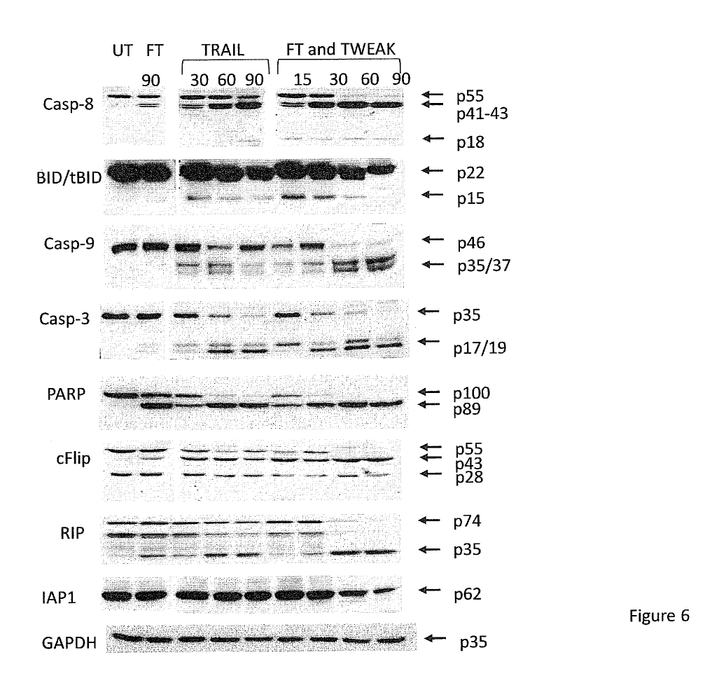

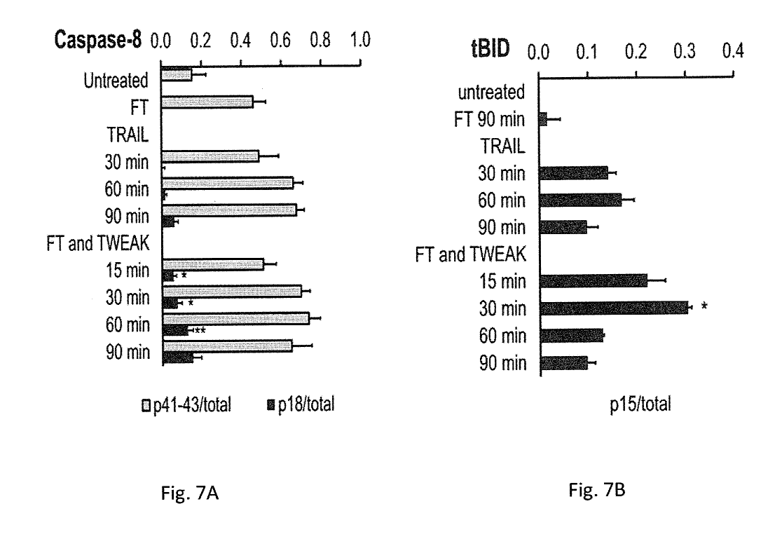

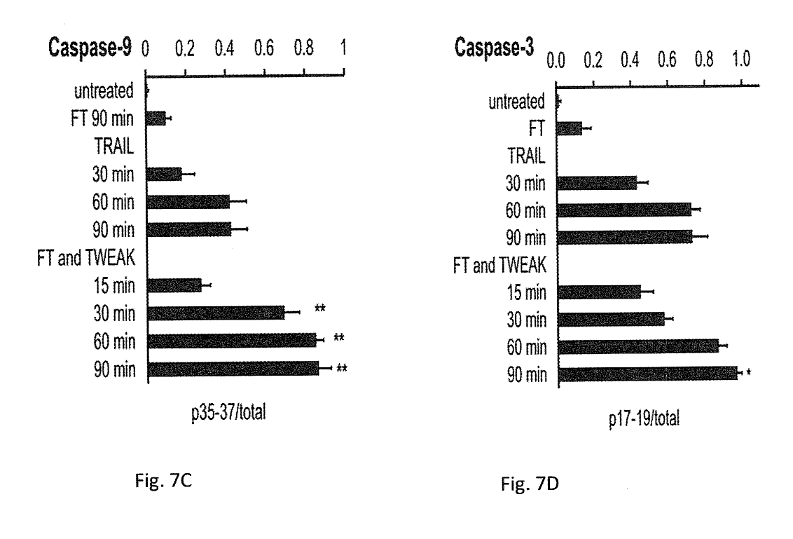

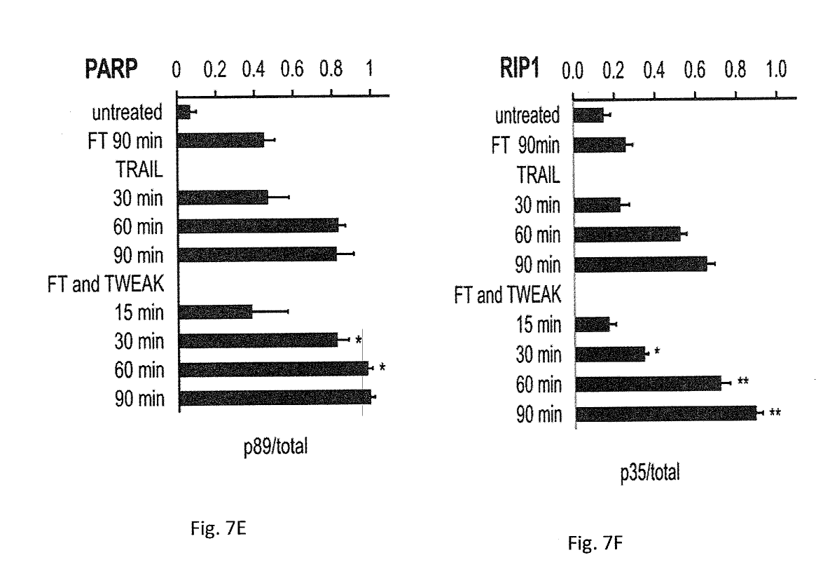

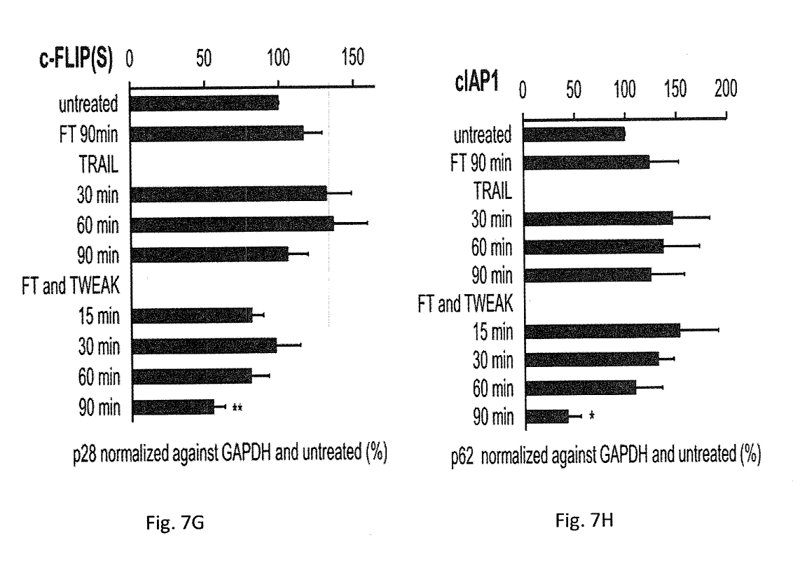

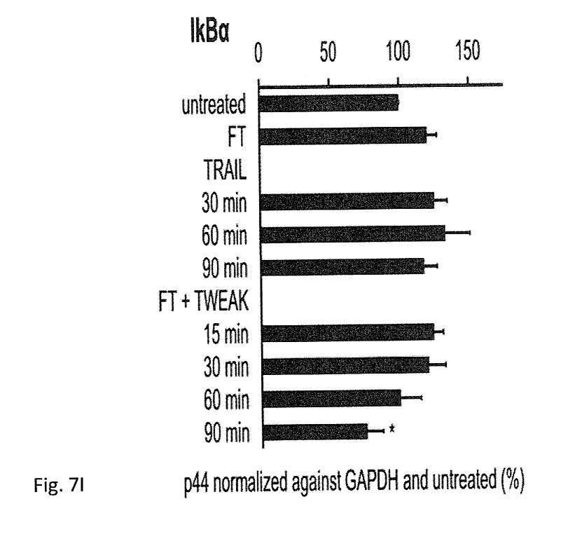

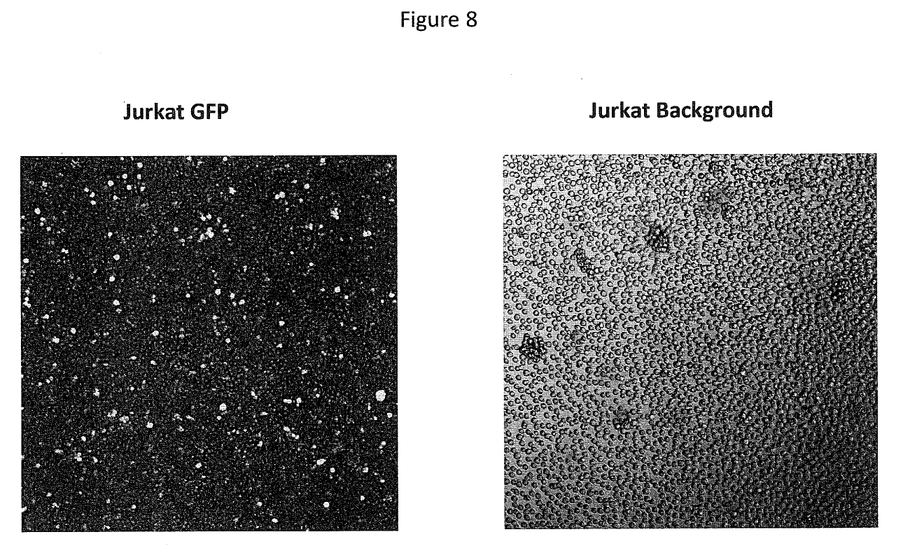

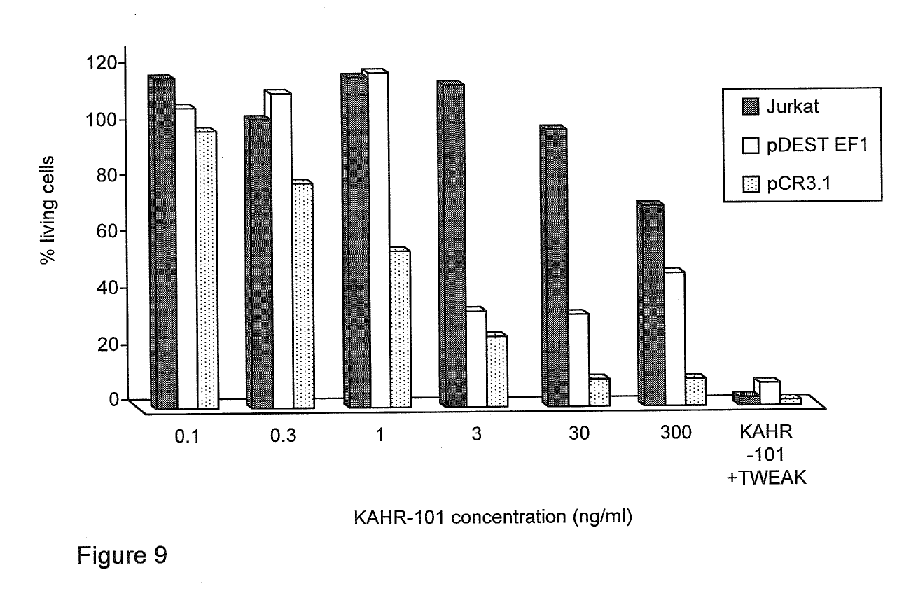

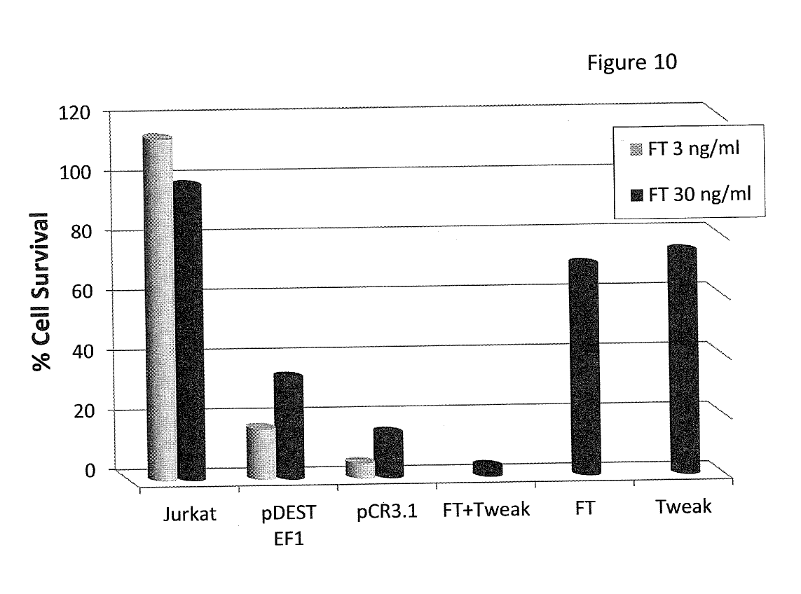

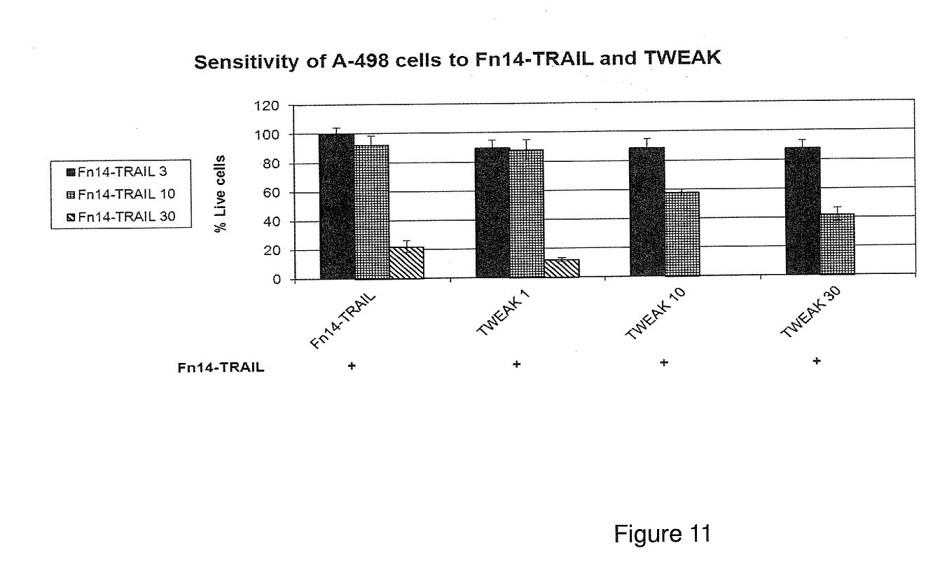

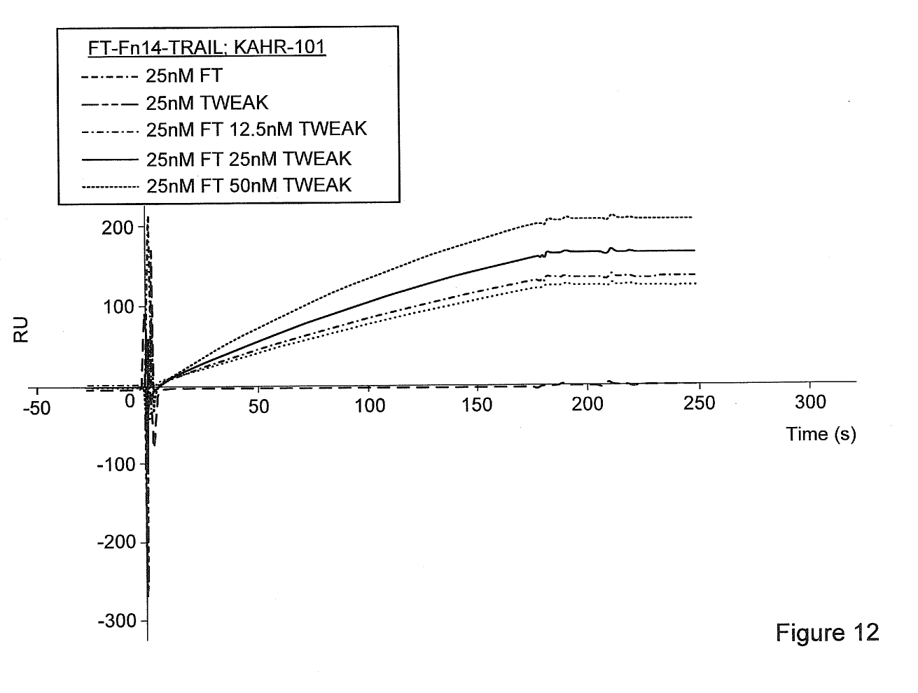

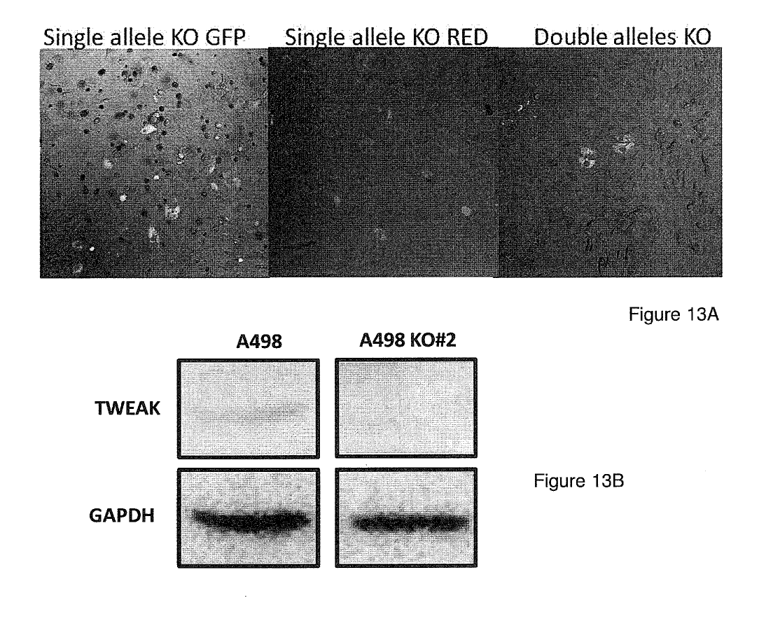

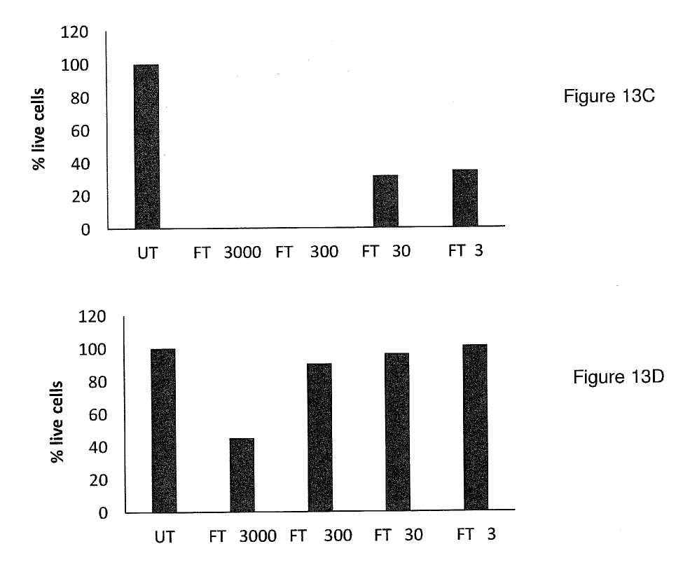

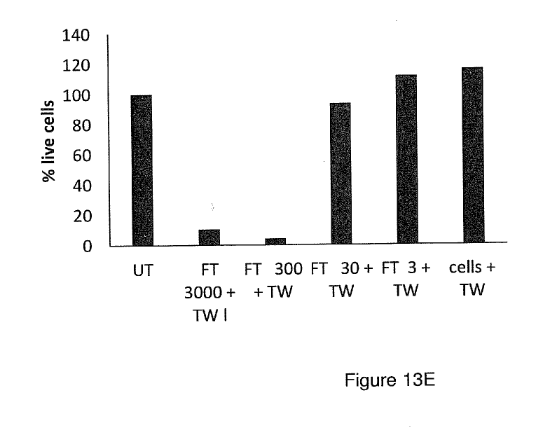

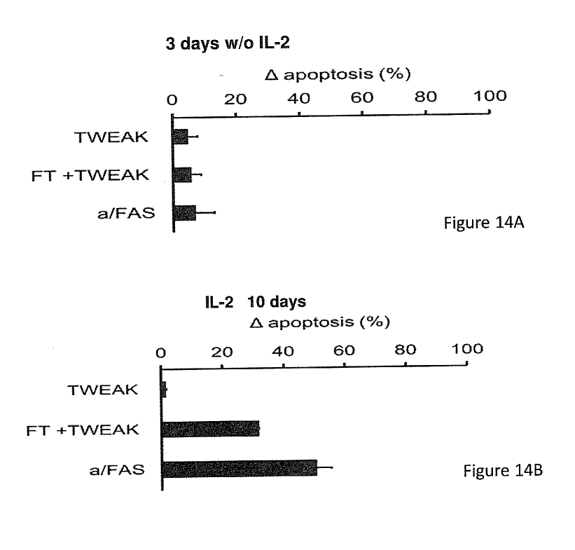

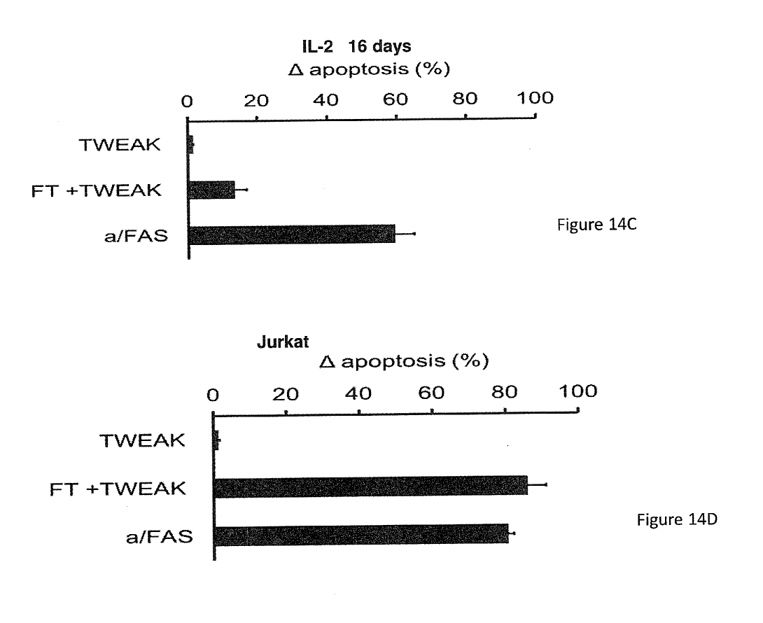

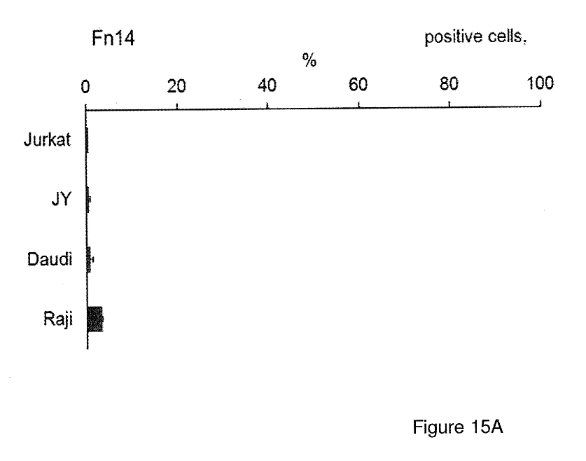

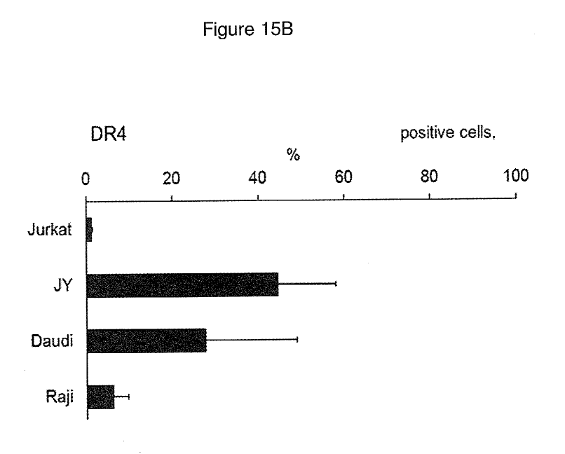

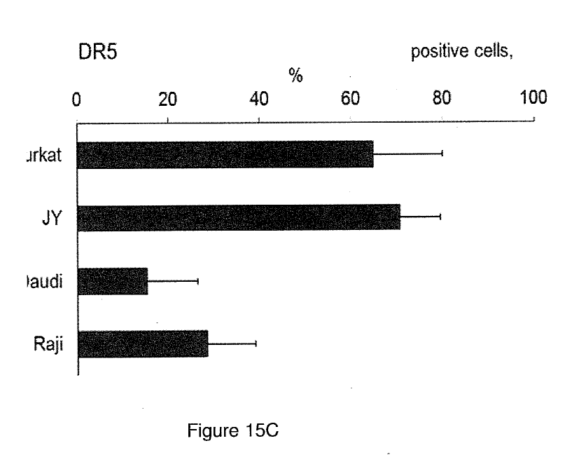

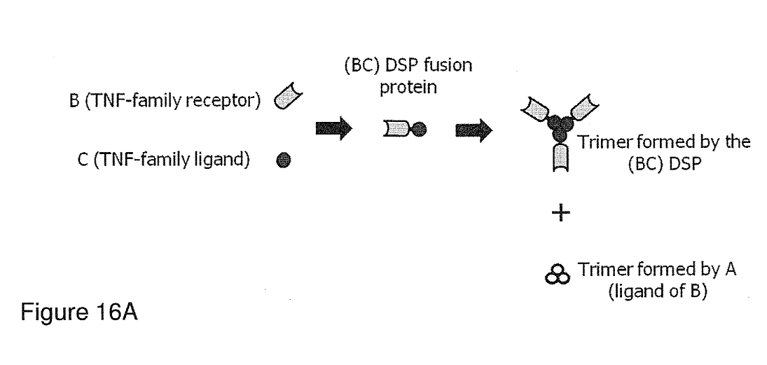

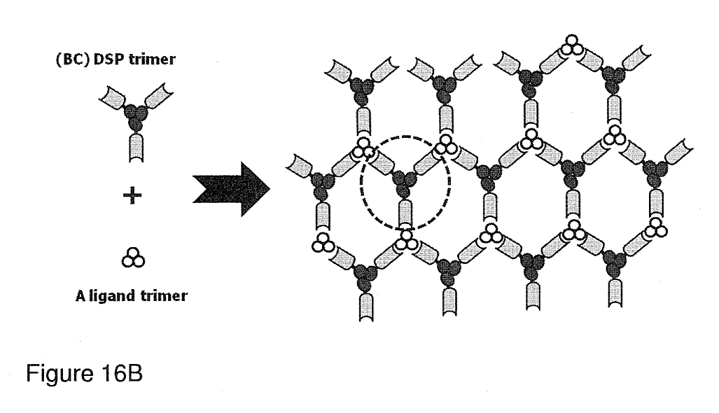

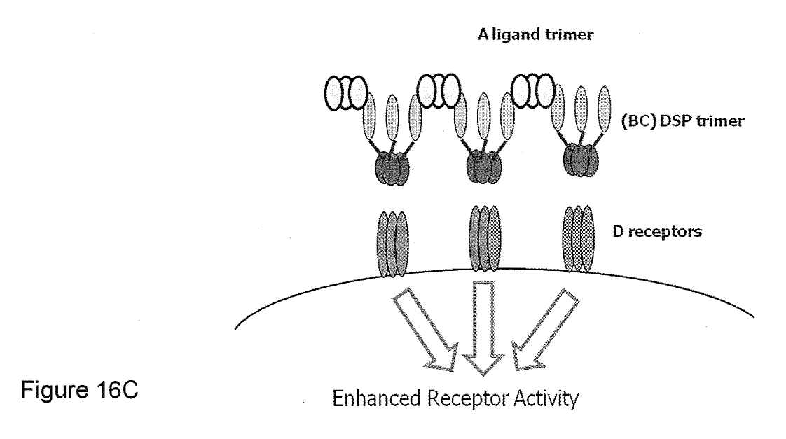

Jurkat cells were incubated for 30 minutes with 20 μg/mL neutralizing mAbs against TRAIL (FIG. 2A), DR5 (FIG. 2B), TWEAK (FIG. 2C), Fn14 (FIG. 2D) or related isotype controls. Fn14-TRAIL (25 ng/mL), Fn14-TRAIL (25 ng/mL) and TWEAK (100 ng/mL) or TWEAK (100 ng/mL) alone were then added to the cells. Percentage of Annexin V-positive cells was examined in all groups by flow cytometry 24 h later. The average of two independent experiments±SE is shown.

FIGS. 3A-B

Fn14-TRAIL Fusion Protein Modified by TWEAK Binds Jurkat Cells Via TRAIL Receptor DR5

Jurkat cells were left untreated or incubated at 4° C. for 30 minutes with TRAIL, Fn14-TRAIL, or Fn14-TRAIL and TWEAK in combination (TWEAK 100 ng/mL, the rest proteins 250 ng/mL). Unbound proteins were then removed by washing; and cells were stained with anti-DR5 (FIG. 3A) or anti-Fn14 (FIG. 3B) fluorochrome-labeled antibodies and examined by flow cytometry for the expression of the relevant markers. In (FIG. 3B) same experiments were performed in the presence of anti-DR5 blocking antibody. The results are presented as average percentage ±SE of 4 experiments. **P<0.001 versus untreated.

FIGS. 4A-E

Treatment of Jurkat Cells with the Combination of Fn14-TRAIL and TWEAK Caused Quicker Apoptosis Induction than Treatment with Each of TRAIL or Fn14-TRAIL Alone, Although all Three Ligands Activated Both Caspase-8 and Caspase-9 Apoptotic Pathways

FIGS. 4A-C. Kinetics of Jurkat cells apoptosis induction by Fn14-TRAIL (FIG. 4A), TRAIL (FIG. 4C) or Fn14-TRAIL in combination with TWEAK (FIG. 4B) was evaluated during the first four hours of incubation. Fn14-TRAIL and Trail were added to cell culture at concentrations 250 ng/ml, while TWEAK was added at the concentration 100 ng/ml. Each 30 min samples of incubated cells were stained with annexin V-FITC Kit and analyzed by flow cytometry. The results are presented as an average percentage±SE of early (FITC⁺PI⁻) and total apoptotic cells over percentage of early or total apoptotic cells in untreated sample. The average of three independent experiments ±SE is shown.

FIG. 4D, FIG. 4E. Effects of caspase-8 and caspase-9 inhibitors on cell death caused by TRAIL, Fn14-TRAIL or Fn14-TRAIL in combination with TWEAK

Jurkat cells were incubated for one hour with 40 μM universal caspase inhibitor Z-VAD-FMK, caspase-8 inhibitor Z-IETD-FMK, caspase-9 inhibitor Z-LEHD-FMK or an equivalent amount of DMSO before 24 h incubation with the indicated above concentrations of Fn14-TRAIL, TWEAK, Fn14-TRAIL and TWEAK (FIG. 4D), or TRAIL (FIG. 4E). The incubated cells were stained for apoptosis and analyzed by flow cytometry. The results are presented as the average percentage of apoptotic cells from three independent experiments±SE.

FIG. 5

Soluble TWEAK (sTWEAK) Addition to Fn14-TRAIL Results in the Formation of a Larger Complex

Soluble TWEAK (50 ng/lane), Fn14-TRAIL (70 ng/lane), and their mixture were separated by native gel electrophoresis and immunoblotted with anti-TRAIL (Abcam) or anti-TWEAK (Cell Signaling) antibodies. 1: TWEAK, 2: Fn14-TRAIL; 3: TWEAK+Fn14-TRAIL

FIG. 6

Western Blotting Detection of Apoptosis-Related Proteins Activated in Jurkat Cells by Treatment with Fn14-TRAIL, TRAIL or Fn14-TRAIL and TWEAK

Jurkat cells (10⁶/mL) were left untreated or were cultured in the presence of Fn14-TRAIL (250 ng/mL), TRAIL (250 ng/mL) or Fn14-TRAIL (250 ng/ml) and TWEAK (100 ng/mL). Cell lysates were prepared at the indicated times and tested by Western blotting for the expression of specified proteins. A representative immunoblot of 3-4 independent experiments for each protein is shown.

FIGS. 7A-I

Western Blotting Data Analysis Demonstrates Earlier and Stronger Activation of Intracellular Pro-Apoptotic Molecular Pathways and Inhibition of Anti-Apoptotic Molecular Pathways by TWEAK and Fn14-TRAIL Compared to Those Measured in the Presence of TRAIL

Western blotting data normalized against GAPDH were used for the analysis of the kinetics of pro-apoptotic proteins expression detected in lysates of cells incubated with Fn14-TRAIL, TRAIL or Fn14-TRAIL and TWEAK (see reference to FIG. 6). Most charts represent data as average ±SE of the cleaved (activated) protein fraction of total protein detected in the lysate by a specified antibody. Thus, p18/total caspase-8 fraction was calculated as p18/(p18+p41-43+p55), while p 34-35/total caspases-9 fraction was calculated as p34-35/(p34-35+p45). Otherwise, when changes in the protein expression were not accompanied by the protein cleavage, the results were normalized both against GAPDH and against untreated control. Three or four independent Western blots were analyzed for each protein.

FIG. 8 Jurkat Cell Transfection

Jurkat cell transfection efficiency was evaluated by co-transfection of the TWEAK expressing plasmids with a GFP producing plasmid.

FIG. 9 Effect of TWEAK Expression on Cell Survival

Jurkat naïve and Jurkat cells transfected with Human Full TWEAK encoding DNA were incubated for 24 hours in the presence of Fn14-TRAIL (FT) at different concentrations (0.1, 0.3, 1, 3, 30, and 300 ng/ml) and cell survival was evaluated by MTS assay. A combination of Fn14-TRAIL and recombinant soluble TWEAK (30 ng/ml each) was used as positive control.

Jurkat-naïve cells; pDEST-EF1 and pCR3.1 refer to the TWEAK transfected Jurkat cells.

FIG. 10 Effect of Soluble TWEAK on Cell Survival

Jurkat naïve cells were incubated for 24 hours with 3 ng/ml or 30 ng/ml Fn14-TRAIL with the addition of conditioned media collected from TWEAK transfected or naïve Jurkat cells cultures. Cell survival was evaluated by MTS. A combination of Fn14-TRAIL and recombinant soluble TWEAK (30 ng/ml each), 30 ng/ml Fn14-TRAIL alone or 30 ng/ml recombinant soluble TWEAK were used as controls.

Jurkat refers to naïve cells and pDEST-EF1 or pCR3.1 refer to the TWEAK transfected Jurkat cells conditioned media added to the cultured naïve Jurkat cells.

FIG. 11 TWEAK Potentiates Fn14-TRAIL Inhibitory Effect on Renal Cell Carcinoma Cells

A498 (RCC cell line) cells were incubated for 24h in the presence of increasing concentrations of Fn14-TRAIL in the presence of increasing concentrations of of soluble TWEAK. Cell survival was tested by MTS assay.

FIG. 12 TWEAK Improves Fn14-TRAIL Binding to the TRAIL-Receptor DR5

Bia-core was performed in order to asses Fn14-TRAIL binding to the TRAIL-receptor DR5. Fn14-TRAIL alone TWEAK alone or Fn14-TRAIL together with increasing amounts of TWEAK were tested.

FIGS. 13A-E: TWEAK Expression in Target Cells is Enhancing Fn14-TRAIL's Killing Effect

TWEAK KO were made from WT A498 RCC cell line (FIG. 13A). WT and TWEAK knock out A498 cells (after sorting) lysats were immuno-blotted with anti TWEAK Abs (FIG. 13B). WT (FIG. 13C) and TWEAK knock out A498 (FIG. 13D) and cells were incubated with Fn14-TRAIL for 24h. TWEAK knockout A498 cells were also incubated with Fn14-TRAIL with the addition of soluble TWEAK (FIG. 13E) for 24h. Cell viability was measured by MTS.

FIGS. 14A-D

Sensitivity of Healthy Human T-Cell Blasts to Apoptosis by Fn14-TRAIL Plus TWEAK Combination and to Anti-FAS Monoclonal Antibody

Peripheral blood lymphocytes (PBL) from a healthy donor were activated in vitro by 0.3 μg/mL of anti-human CD3 mAb (OKT3). Day +3 T cell blasts (1*10⁶ cells/mL) were cultured for 24h with TWEAK (100 ng/mL), Fn14-TRAIL (250 ng/ml) and TWEAK (100 ng/mL) or cytotoxic anti-FAS antibody CH11 (100 ng/mL), for apoptosis induction (FIG. 14A) or left in culture with IL-2 (10 U/ml) for additional 10 (FIG. 14B) or 16 (FIG. 14C) days. At the end of culture T-cell blasts were exposed to TWEAK, Fn14-TRAIL plus TWEAK, or anti-FAS antibody as indicated in FIG. 14A. Treated cells were stained for apoptosis evaluation and analyzed by flow cytometry. The results are given as percentage of apoptotic cells over control incubated for the last 24 h only with medium and IL-2. (FIG. 14D) Percentage of apoptotic Jurkat cells detected after 24 h incubation with the same amount of Fn14-TRAIL and TWEAK or anti-Fas antibody. The average of two independent experiments±SE is shown.

FIGS. 15A-C

Comparison of Cell Surface Expression of TRAIL Receptors DR4 and DR5, and TWEAK Receptor Fn14 on Lymphoblasts Derived from Jurkat, J Y, Daudi and Raji

Lymphoblasts derived from cultured cell lines were stained with PE-conjugated anti-Fn14, anti-DR4, or anti-DR5 antibodies and analyzed by flow cytometry. The results are presented as percentage of specific marker positive cells±SE. Bars on Y axes are arranged in the order of decreasing susceptibility to apoptosis by Fn14-TRAIL and TWEAK, with Jurkat cells being most susceptible to apoptosis.

FIGS. 16A-16C

A non-limiting schematic representation of the suggested clustering model, and its potential effect on the activities of the participating molecules.
A) A DSP fusion protein, linking the extracellular portions of a TNF-family receptor (B) and a TNF-family ligand (C) can form a homo-trimer due to the natural ability of TNF-family ligands to trimerize. The (BC) DSP trimer, can bind a different TNF-family ligand trimer (A) [the ligand of (B)].
B) The interaction of the (BC) DSP trimer with an (A) trimer, can form a cluster of (A)+(BC)
C) The cluster of (A)+(BC) would likely form at locations in which (A) concentrations are high, for example, in the body if (A) is presented in higher amount in a diseased tissue or organ or their environment as compared to a healthy organ or tissue, will block the activity of (A) and induce the activity of (C) via clustering of (D) receptors on cell membranes or positioning the (C) ligands in a way that will enhance their binding to (D).

### DESCRIPTION OF DETAILED EMBODIMENTS OF THE INVENTION

Surprisingly, the present inventors have found that specific fusion proteins may be advantageously administered to subjects suffering from inflammatory, immune related or cancerous diseases, depending upon the presence of another TNF-family ligand and/or TNF-receptor. Such other TNF-family ligand may also optionally be administered with the fusion protein, as part of a stabilized composition.

The terms “DSP” and “fusion protein” are used herein interchangeably.

The present inventors have found that this combination of a fusion protein as described herein, plus an additional TNF-family ligand, may optionally be selected as follows. Consider two TNF-family ligand/receptor pairs, labeled A/B and C/D, respectively. A and C are the ligands; B and D are the receptors. The receptors are different from each other, as are the ligands. Furthermore, ligand A does not bind to receptor D, nor does ligand C bind to receptor B. A fusion protein may optionally be constructed from the ECD (extracellular domain) of receptor B and ligand C. According to at least some embodiments, a diagnostic method to determine whether this fusion protein may be advantageously administered to a subject would therefore comprise detecting a level of ligand A in the subject, for example and without limitation globally (for example in a blood, Cerebrospinal Fluid (CSF), synovial fluid, saliva or urine or any other body fluids or excretions of a subject, or locally, for example by testing specific diseased tissues.

According to at least some embodiments, a stabilized composition may optionally comprise the above fusion protein, comprising the ECD of receptor B and ligand C, plus a stabilizing amount of ligand A.

A non-limiting example of such a group of TNF-family ligand/receptor pairs is TWEAK (ligand A)/Fn14 (receptor B) and TRAIL (ligand C)/TRAIL-receptor (receptor D). Other examples of possible ligand/receptor pairs are given in Table 1.

TWEAK/TRAIL Signaling Axes and Fusion Proteins

The present invention provides, in one aspect, a fusion protein which acts on the TWEAK and TRAIL signaling axes, for example a fusion protein having a first domain that comprises a polypeptide that binds to a TWEAK ligand; and a second domain that comprises a polypeptide that binds to the TRAIL receptor; as a non-limiting example of the above DSP.

In particular, the first domain is a polypeptide that has the capacity to interfere with TWEAK'S ability to trigger through its Fn14 receptor, and the second domain is a polypeptide that can direct signals through cognate receptors on T cells or other cells bearing the TRAIL receptor.

Suitable first domains in the context of the TWEAK and TRAIL signaling axes include, for example, the Fn14 protein, variants or derivatives of the wild-type Fn14 protein, or other polypeptides or proteins specifically tailored to bind TWEAK and prevent this ligand from signaling through its Fn14 receptor, such as antibodies that bind to TWEAK, parts of antibodies that bind to TWEAK, and lipocalin derivatives engineered to bind to TWEAK. Preferably, the first domain of the fusion protein of this embodiment is at least a portion of the extracellular domain of the Fn14 protein, specifically that portion of the extracellular domain which is necessary for binding to the TWEAK ligand and interfering with its ability to bind and trigger a membrane-bound Fn14 receptor. Variants of the wild-type form of the extracellular domain, or the portion of the extracellular domain responsible for TWEAK binding, are also included in the present invention, so long as the variant provides a similar level of biological activity as the wild-type protein.

Accordingly, the term “polypeptide that binds to a TWEAK ligand” as used herein includes the Fn14 protein; the extracellular domain of the Fn14 protein; a polypeptide which is at least a portion of the extracellular domain of the Fn14 protein, the portion responsible for binding to a TWEAK ligand; antibodies or parts of antibodies to TWEAK; lipocalin derivatives; and variants and/or derivatives of any of these. The term “Fn14” is understood to embrace polypeptides corresponding to the complete amino acid sequence of the Fn14 protein, including the cytoplasmic, transmembrane and extracellular domains, as well as polypeptides corresponding to smaller portions of the protein, such as the extracellular domain, or a portion of the extracellular domain. In one embodiment the first domain of the Fn14/TRAIL fusion protein is at least a portion of the extracellular domain of the human Fn14 protein.

Suitable second domains in the context of the TWEAK and TRAIL signaling axes include, for example, the TRAIL protein itself, variants or derivatives of the TRAIL protein, or other polypeptides or proteins that are specifically designed to inhibit activation of T cells or other cells and/or induce apoptosis through the TRAIL receptor, or induce or inhibit any other TRAIL activity, such as agonistic anti-TRAIL Ab, and variants and/or derivatives of any of these.

Preferably, the second domain of the fusion protein in this embodiment is at least a portion of the extracellular domain of the TRAIL protein, specifically that portion which is necessary for binding to a TRAIL receptor. Variants of the wild-type form of the extracellular domain of the TRAIL protein, or the portion of the extracellular domain responsible for TRAIL receptor binding, are also included in the present invention, so long as the variant provides a similar level of biological activity as the wild-type protein and in some embodiments is capable of forming trimers.

Accordingly, the term “polypeptide that binds to a TRAIL receptor” as used herein includes the TRAIL protein; the extracellular domain of the TRAIL protein; a polypeptide which is at least a portion of the extracellular domain of the TRAIL protein, the portion responsible for binding to a TRAIL receptor; antibodies (and parts of antibodies) to a TRAIL receptor; and variants and/or derivatives of any of these. The term “TRAIL” is understood to embrace polypeptides corresponding to the complete amino acid sequence of the TRAIL protein, including the cytoplasmic, transmembrane and extracellular domains, as well as polypeptides corresponding to smaller portions of the protein, such as the extracellular domain, or a portion of the extracellular domain. In one embodiment the second domain of Fn14-TRAIL fusion protein is at least a portion of the extracellular domain of the human TRAIL protein.

In one embodiment, the present invention comprises a Fn14/TRAIL fusion protein. In another embodiment, the term “Fn14/TRAIL fusion protein” refers to the specific fusion protein identified by SEQ ID NO. 1:

MRALLARLLLCVLVVSDSKGEQAPGTAPCSRGSSWSAD
LDKCMDCASCRARPHSDFCLGCAAAPPAPFRLLWRGPQ
RVAAHITGTRGRSNTLSSPNSKNEKALGRKINSWESSRSG
HSFLSNLHLRNGELVIHEKGFYYIYSQTYFRFQEEIKENTK
NDKQMVQYIYKYTSYPDPILLMKSARNSCWSKDAEYGLY
SIYQGGIFELKENDRIFVSVTNEHLIDMDHEASFFGAFLVG

1. The signal peptide of the human Urokinase protein is underlined i.e. M R A L L A R L L L C V L V V S D S K G (SEQ ID NO. 2)
2. The extracellular domain of the human Fn14 is bold i.e. E Q A P G T A P C S R C S S W S A D L D K C M D C A S C R A R P H S D F C L G C A A A P P A P F R L L W (SEQ ID NO. 3)
3. The extracellular domain of the human TRAIL in regular font i.e. R G P Q R V A A H I T G T R G R S N T L S S P N S K N E K A L G R K I N S W E S S R S G H S F L S N L H L R N C E L V I H E K G F Y Y I Y S Q T Y F R F Q E E I K E N T K N D K Q M V Q Y I Y K Y T S Y P D P I L L N I K S A R N S C W S K D A E Y G L Y S I Y Q G G I F E L K E N D R I F V S V T N E H L I D M D H E A S F F G A F L V G (SEQ ID NO. 4).

In additional embodiments, the Fn14/TRAIL fusion protein is a variant and/or derivative of the amino acid sequence shown in SEQ ID NO. 1. A number of such variants are known in the art, as for example in U.S. Pat. No. 8,039,437, hereby incorporated by reference as if fully set forth herein.

In yet an additional aspect of the present invention, the TRAIL component of any of the fusion proteins described herein can be substituted with another inhibitory protein, i.e. a protein which prevents activation of an immune response and/or induces apoptosis in T cells or other cell types, such as B cells, natural killer (NK) cells, NKT cells, lymphoid progenitor cells, dendritic cells, monocytes/macrophages, tissue-based macrophage lineage cells with antigen-presenting capacity, and any one of a number of non-professional antigen-presenting cells, for example, endothelial cells. Examples of inhibitory proteins are described herein.

In one embodiment, the fusion proteins of the present invention inhibit activation of the immune system by preventing or reducing proliferation and differentiation of myelin-specific T cells. In some embodiments the fusion proteins of the present invention inhibit production of pro-inflammatory cytokines and chemokines, such as IL-6, IL-8, RANTES, IP-10, and MCP-1, or inhibit potentiation of other cytokines/chemokines, such as TNF-alpha and IL-1beta; or inhibit induction of matrix metalloproteinases such as MMP-1 and MMP-9; or inhibit prostaglandin E2 secretion from fibroblasts and synoviocytes. The present invention embraces inhibition/down-regulation of any and all cytokines that are either promoted by TWEAK ligand or down-modulated by the TRAIL ligand.

In other embodiments the fusion proteins of the present invention inhibit autoreactive T cell proliferation, autoreactive antibody production, and inflammatory reactions.

In additional embodiments, the fusion proteins of the present invention reduce inflammation as determined in in vitro and in vivo assays that measure inhibition of pro-inflammatory cytokine and chemokine production and/or elevation of anti-inflammatory cytokine production, in in vivo model systems of inflammation, such as autoimmune disease models, for example, EAE and collagen-induced arthritis, and delayed-type hypersensitivity and other models in which pro-inflammatory agents are introduced locally or systemically into animals. In these in vivo models, inflammation is assessed by histological examination of inflamed tissues, isolation of inflammatory cells from diseased tissues, and measurement of disease manifestations in affected animals. The fusion proteins of the present invention, in other embodiments, inhibit the proliferation, differentiation and/or effector function of pathogenic T cells such as autoreactive CD4+ T cells and CD8+ T cells and other pathogenic immune cells such as B cells, natural killer (NK) cells, NKT cells, lymphoid progenitor cells, dendritic cells, monocytes/macrophages; induce apoptosis in pathogenic immune cells; promote generation of immune cells with regulatory properties (such as CD4+ CD25+ regulatory T cells, Tr1 cells, CD8+, NK NKT, and dendritic cells with immuno-inhibitory activities); decrease permeability of the blood-brain barrier, and thereby restrict access of inflammatory cells to the CNS; decrease access of inflammatory cells to other disease sites, and decrease angiogenesis associated with inflammation.

Fn14

Fn14 is a plasma membrane-anchored protein and a TNFR (TNF receptor) superfamily member of 129 amino acids in length (Swiss Prot Accession number Q9CR75 (mouse) and Q9NP84 (human) Two variants of Fn14 are known, identified by Swiss Prot. Isoform ID Nos. Q9NP84.1 and Q9NP84.2 (NCBI accession numbers are NP.sub.-057723 and BAB17850, respectively). The Fn14 sequence has also been determined in many other species, including Xenopus laevis (NCBI Accession No. AAR21225) and rat (NCBI Accession No. NP.sub.-851600].

Most TNFR superfamily members contain an extracellular domain that is structurally characterized by the presence of one to six cysteine-rich domains (CRDs). The typical CRD is approximately 40 amino acids in length and contains six conserved cysteine residues that form three intrachain disulphide bridges. The CRD itself is typically composed of two distinct structural modules.

Fn14 is a Type I transmembrane proteins that contains a 53-amino-acid extracellular domain, amino acids 28-80, with one CRD. Certain charged amino acid residues within this CRD have been shown to be particularly critical for an effective TWEAK-Fn14 interaction. Brown, S. A. et al., Tweak binding the Fn14 cysteine-rich domain depends on shared residues located in both the A1 and D2 modules, J. Biochem. 397: 297-304 (2006), incorporated herein by reference.

Based on the information provided in the Brown et al. article, for example, one skilled in the art can determine which variants of the Fn14 protein will retain TWEAK binding activity and which ones will not. For example, several specific variants prepared by site-specific mutations at positions that were not evolutionarily conserved were found to have TWEAK binding activity. In contrast, at least three amino acids in the CRD region were critical for TWEAK binding. By comparing the amino acid sequences of the Fn14 protein in a variety of species one can determine which amino acid positions are not highly conserved, and would be good candidates for substitution/addition/deletion. Substitutions/deletions/additions in highly conserved regions, particularly in the TNFR homology region, would not be considered likely candidates for preparation of variants according to the present invention.

TRAIL

TRAIL is a Type II membrane protein having 291 amino acids and has been sequenced in a number of species, including, but not limited to, mouse: Swiss Prot. Accession No. P50592: human: Swiss Prot. Accession No. P50591, Rattus norvegicus: NCBI Accession NP.sub.-663714; Siniperca Chuatsi (Chinese Perch): NCBI Accession AAX77404; Gallus Gallus (Chicken): NCBI Accession BAC79267; Sus Scrofa (Pig): NCBI Accession NP.sub.-001019867; Ctenopharyngodon Idella (Grass Carp): NCBI Accession AAW22593; and Bos Taurus (Cattle): NCBI Accession XP.sub.-001250249.

The extracellular domain of TRAIL comprises amino acids 39-281, and the TNF domain responsible for receptor binding is amino acid 121-280, based on TNF homology models. The portion of the protein that is particularly important for conferring activity has been identified. See, e.g., “Triggering cell death: The crystal structure of Apo2L/TRAIL in a complex with death receptor”, Hymowitz S G, et al., Am. Mol. Cell. 1999 October; 4(4):563-71), incorporated herein by reference, which reports the most important amino acids for TRAIL binding to its receptor and activity are amino acids around the zinc area such as amino acids (191-201-205-207-236-237) and amino acids (150-216), incorporated herein by reference. See also 1) Krieg A et al 2003 Br. J of Cancer 88: 918-927, which describes two human TRAIL variants without apoptotic activity, TRAIL-gamma and TRAIL beta; 2) “Enforced covalent trimerization increases the activity of the TNF ligand family members TRAIL and CD95L”, D Berg et al., Cell death and differentiation (2007) 14, 2021-2034; and 3) “Crystal Structure of TRAIL-DR5 complex identifies a critical role of the unique frame insertion in conferring recognition specificity”, S. Cha et al., J. Biol. Chem. 275: 31171-31177 (2000), all incorporated herein by reference.

TRAIL is known to ligate two types of receptors: death receptors triggering TRAIL-induced apoptosis and decoy receptors that possibly inhibit this pathway. Four human receptors for TRAIL have been identified, including TRAILR1, TRAILR2, TRAILR3 and TRAILR4. TRAIL can also bind to osteoprotegerin (OPG). Binding to each of these receptors has been well-characterized. See, e.g., “The TRAIL apoptotic pathway in cancer onset, progression and therapy”, Nature Reviews Cancer Volume 8 (2008) 782-798.

TWEAK-Related Conditions or Diseases

According to at least some embodiments, TWEAK-related conditions include but are not limited to various cancers as described below, acute cardiac injury, chronic heart failure, cardiomyopathy (including without limitation non-inflammatory dilated cardiomyopathy), congestive heart failure, liver epithelial cell hyperplasia, hepatocyte death, hepatocyte vacuolation, other liver injuries, bile duct conditions, including bile duct hyperplasia, liver inflammatory disease, inflammatory kidney conditions, such as multifocal inflammation, non-inflammatory kidney conditions such as tubular nephropathy, tubular hyperplasia, glomerular cysts, glomerular nephropathy, Glomerulonephritis, Alport Syndrome, kidney tubular vacuolation, kidney hyaline casts, various fibrotic diseases (including without limitation liver fibrosis, kidney fibrosis and/or lung fibrosis) and inflammatory lung disease, ovarian cancer, colorectal cancer, head and neck squamous cell carcinoma (HNSCC), colonic adenocarcinoma, hepatocellular carcinoma, kidney cancer, stomach cancer, breast cancer, squamous cell carcinoma, esophageal cancer, pancreatic cancer, cervical cancer, colorectal cancer, glioma, head and neck cancer, liver cancer, melanoma, prostate cancer, skin cancer, testis cancer, thyroid cancer, urothelial cancer, Hodgkin lymphoma metastatic neuroblastoma, glioblastoma, astrocytoma and astrocyte brain tumor, lung carcinoma, pancreas adenocarcinoma, ovarian cystadenocarcinoma, cervical squamous carcinoma, prostate adenocarcinoma, squamous cell carcinoma, keratinocyte carcinoma, metastatic malignant melanoma, osteosarcoma, or metastatic choriocarcinoma

In some embodiments of the invention the diseases to be treated by a fusion protein comprising Fn14 or the ECD thereof, such as for example, Fn14-TRAIL are: seborrheic keratosis, systemic lupus erythematosus (SLE), Lupus Nephritis, Rheumatoid Arthritis (RA), inflammatory bowel disease (IBD) as ulcerative colitis and Crohn's disease, multiple sclerosis, Parkinson's disease, amyotrophic lateral sclerosis (ALS), atopic dermatitis, psoriasis vulgaris, psoriatic arthritis, urticarial vasculitis, myocardial infarction, proliferative diabetic retinopathy, or acute ischemic stroke

In some embodiments of the invention the diseases to be treated by a fusion protein comprising Fn14 or the ECD thereof, such as for example, Fn14-TRAIL are: seborrheic keratosis, Inflammatory bowel disease (IBD) as ulcerative colitis and Crohn's disease, Lupus Nephritis, Parkinson's disease, amyotrophic lateral sclerosis (ALS), psoriasis vulgaris, psoriatic arthritis, myocardial infarction, proliferative diabetic retinopathy, or retinopathy caused by any other condition (for example, hypertension, radiation, sickle cell disease and the like), or acute ischemic stroke.

In some embodiments of the invention the diseases to be treated by a fusion protein comprising Fn14 or the ECD thereof, such as for example, Fn14-TRAIL are: testis cancer, urothelial cancer, Hodgkin lymphoma, Squamous cell carcinoma, keratinocyte carcinoma or osteosarcoma.

Protein Chemical Modifications

In the present invention any part of a protein of the invention may optionally be chemically modified, i.e. changed by addition of functional groups. For example the side amino acid residues appearing in the native sequence may optionally be modified, although as described below alternatively other parts of the protein may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible.

The amino acid of any of the sequence regions of the molecule can optionally be modified according to any one of the following exemplary types of modification (in the peptide conceptually viewed as “chemically modified”). Non-limiting exemplary types of modification include carboxymethylation, acylation, phosphorylation, glycosylation or fatty acylation. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, when referring to a protein or peptide according to the present invention, refers to a protein or peptide where at least one of its amino acid residues is modified either by natural processes, such as processing or other post-translational modifications, or by chemical modification techniques which are well known in the art. Examples of the numerous known modifications typically include, but are not limited to: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

Other types of modifications optionally include the addition of a cycloalkane moiety to a biological molecule, such as a protein, as described in PCT Application No. WO 2006/050262, hereby incorporated by reference as if fully set forth herein. These moieties are designed for use with biomolecules and may optionally be used to impart various properties to proteins.

Furthermore, optionally any point on a protein may be modified. For example, pegylation of a glycosylation moiety on a protein may optionally be performed, as described in PCT Application No. WO 2006/050247, hereby incorporated by reference as if fully set forth herein. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

By “PEGylated protein” is meant a protein, or a fragment thereof having biological activity, having a polyethylene glycol (PEG) moiety covalently bound to an amino acid residue of the protein.

By “polyethylene glycol” or “PEG” is meant a polyalkylene glycol compound or a derivative thereof, with or without coupling agents or derivatization with coupling or activating moieties (e.g., with thiol, triflate, tresylate, azirdine, oxirane, or preferably with a maleimide moiety). Compounds such as maleimido monomethoxy PEG are exemplary or activated PEG compounds of the invention. Other polyalkylene glycol compounds, such as polypropylene glycol, may be used in the present invention. Other appropriate polyalkylene glycol compounds include, but are not limited to, charged or neutral polymers of the following types: dextran, colominic acids or other carbohydrate based polymers, polymers of amino acids, and biotin derivatives.

Altered Glycosylation Protein Modification

Proteins of the invention may be modified to have an altered glycosylation pattern (i.e., altered from the original or native glycosylation pattern). As used herein, “altered” means having one or more carbohydrate moieties deleted, and/or having at least one glycosylation site added to the original protein.

Glycosylation of proteins is typically either N-linked or O-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to proteins of the invention is conveniently accomplished by altering the amino acid sequence of the protein such that it contains one or more of the above-described tripeptide sequences (for N-linked glycosylation sites). The alteration may also be made by the addition of, or substitution by, one or more serine or threonine residues in the sequence of the original protein (for O-linked glycosylation sites). The protein's amino acid sequence may also be altered by introducing changes at the DNA level.

Another means of increasing the number of carbohydrate moieties on proteins is by chemical or enzymatic coupling of glycosides to the amino acid residues of the protein. Depending on the coupling mode used, the sugars may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, CRC Crit. Rev. Biochem., 22: 259-306 (1981).

Removal of any carbohydrate moieties present on proteins of the invention may be accomplished chemically, enzymatically or by introducing changes at the DNA level. Chemical deglycosylation requires exposure of the protein to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259: 52 (1987); and Edge et al., Anal. Biochem., 118: 131 (1981). Enzymatic cleavage of carbohydrate moieties on proteins can be achieved by the use of a variety of endo- and exo-glycosidases as described by Thotakura et al., Meth. Enzymol., 138: 350 (1987).

Pharmaceutical Compositions

The present invention, in some embodiments, features a pharmaceutical composition comprising a therapeutically effective amount of a therapeutic agent according to the present invention. According to the present invention the therapeutic agent could be a polypeptide as described herein. The pharmaceutical composition according to the present invention is further used for the treatment of cancer or an immune related disorder as described herein. The therapeutic agents of the present invention can be provided to the subject alone, or as part of a pharmaceutical composition where they are mixed with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., a polypeptide as described herein, may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the present invention also may include a pharmaceutically acceptable anti-oxidants. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. A pharmaceutical composition according to at least some embodiments of the present invention also may include additives such as detergents and solubilizing agents (e.g., TWEEN 20 (polysorbate-20), TWEEN 80 (polysorbate-80)) and preservatives (e.g., Thimersol, benzyl alcohol) and bulking substances (e.g., lactose, mannitol).

Examples of suitable aqueous and nonaqueous carriers that may be employed in the pharmaceutical compositions according to at least some embodiments of the present invention include water, buffered saline of various buffer content (e.g., Tris-HCl, acetate, phosphate), pH and ionic strength, ethanol, polyols (such as glycerol, propylene glycol, polyethylene glycol, and the like), and suitable mixtures thereof, vegetable oils, such as olive oil, and injectable organic esters, such as ethyl oleate.

Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the present invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

A composition of the present invention can be administered via one or more routes of administration using one or more of a variety of methods known in the art. As will be appreciated by the skilled artisan, the route and/or mode of administration will vary depending upon the desired results. Preferred routes of administration for therapeutic agents according to at least some embodiments of the present invention include intravascular delivery (e.g. injection or infusion), intravenous, intramuscular, intradermal, intraperitoneal, subcutaneous, spinal, oral, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intraperitoneal, intravenous (IV) and intradermal), transdermal (either passively or using iontophoresis or electroporation), transmucosal (e.g., sublingual administration, nasal, vaginal, rectal, or sublingual), administration or administration via an implant, or other parenteral routes of administration, for example by injection or infusion, or other delivery routes and/or forms of administration known in the art. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion or using bioerodible inserts, and can be formulated in dosage forms appropriate for each route of administration. In a specific embodiment, a protein, a therapeutic agent or a pharmaceutical composition according to at least some embodiments of the present invention can be administered intraperitoneally or intravenously.

Compositions of the present invention can be delivered to the lungs while inhaling and traverse across the lung epithelial lining to the blood stream when delivered either as an aerosol or spray dried particles having an aerodynamic diameter of less than about 5 microns. A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected. For the polypeptide compositions disclosed herein and nucleic acids encoding the same, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For polypeptide compositions, generally dosage levels of 0.0001 to 100 mg/kg of body weight daily are administered to mammals and more usually 0.001 to 20 mg/kg. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Generally, for intravenous injection or infusion, dosage may be lower. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

Optionally the polypeptide formulation may be administered in an amount between 0.0001 to 100 mg/kg weight of the patient/day, preferably between 0.001 to 20.0 mg/kg/day, according to any suitable timing regimen. A therapeutic composition according to at least some embodiments according to at least some embodiments of the present invention can be administered, for example, three times a day, twice a day, once a day, three times weekly, twice weekly or once weekly, once every two weeks or 3, 4, 5, 6, 7 or 8 weeks. Moreover, the composition can be administered over a short or long period of time (e.g., 1 week, 1 month, 1 year, 5 years).

Alternatively, therapeutic agent can be administered as a sustained release formulation, in which case less frequent administration is required. Dosage and frequency vary depending on the half-life of the therapeutic agent in the patient. In general, human antibodies show the longest half life, followed by humanized antibodies, chimeric antibodies, and nonhuman antibodies. The half-life for fusion proteins may vary widely. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a polypeptide as disclosed herein preferably results in a decrease in severity of disease symptoms, an increase in frequency and duration of disease symptom-free periods, an increase in lifespan, disease remission, or a prevention or reduction of impairment or disability due to the disease affliction.

One of ordinary skill in the art would be able to determine a therapeutically effective amount based on such factors as the subject's size, the severity of the subject's symptoms, and the particular composition or route of administration selected.

In certain embodiments, the polypeptide compositions are administered locally, for example by injection directly into a site to be treated. Typically, the injection causes an increased localized concentration of the polypeptide compositions which is greater than that which can be achieved by systemic administration. For example, in the case of a neurological disorder like Multiple Sclerosis, the protein may be administered locally to a site near the CNS. In another example, as in the case of an arthritic disorder like Rheumatoid Arthritis, the protein may be administered locally to the synovium in the affected joint. The polypeptide compositions can be combined with a matrix as described above to assist in creating a increased localized concentration of the polypeptide compositions by reducing the passive diffusion of the polypeptides out of the site to be treated.

Pharmaceutical compositions of the present invention may be administered with medical devices known in the art. For example, in an optional embodiment, a pharmaceutical composition according to at least some embodiments of the present invention can be administered with a needles hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in an optional embodiment, a therapeutic composition according to at least some embodiments of the present invention can be administered with a needles hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, C1ORF32 soluble proteins, C1ORF32 ectodomains, C1ORF32 fusion proteins, other proteins or other therapeutic agents according to at least some embodiments of the present invention can be formulated to ensure proper distribution in vivo. For example, the blood-brain barrier (BBB) excludes many highly hydrophilic compounds. To ensure that the therapeutic compounds according to at least some embodiments of the present invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Formulations for Parenteral Administration

In a further embodiment, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., water soluble antioxidants such as ascorbic acid, sodium metabisulfite, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, alpha-tocopherol; and metal chelating agents, such as citric acid, ethylenediamine tetraacetic acid (EDTA), sorbitol, tartaric acid, phosphoric acid), and preservatives (e.g., Thimersol, benzyl alcohol) and bulking substances (e.g., lactose, mannitol). Examples of non-aqueous solvents or vehicles are ethanol, propylene glycol, polyethylene glycol, vegetable oils, such as olive oil and corn oil, gelatin, and injectable organic esters such as ethyl oleate. The formulations may be freeze dried (lyophilized) or vacuum dried and redissolved/resuspended immediately before use. The formulation may be sterilized by, for example, filtration through a bacteria retaining filter, by incorporating sterilizing agents into the compositions, by irradiating the compositions, or by heating the compositions.

Formulations for Topical Administration

Various polypeptides disclosed herein can be applied topically. Topical administration does not work well for most peptide formulations, although it can be effective especially if applied to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers.

### Controlled Delivery Polymeric Matrices

Various polypeptides disclosed herein may also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of polypeptides or nucleic acids encoding the polypeptides, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl Polymer ScL, 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### EXAMPLES

### Fn14-TRAIL Plus TWEAK as Non-Limiting Example of a DSP Fusion Protein and Ligand Combination

As described herein, many different component 1 (such as the ECD of receptor “B”) and component 2 (such as ligand “C”) proteins, as well as functional portions thereof, may optionally form fusion proteins (“B-C”) which may optionally be stabilized and/or administered in the presence of the ligand (“A”) to the receptor of the fusion protein (“B”) and/or the presence of the receptor (“D”) to the ligand of the fusion protein (“C”). Fn14-TRAIL fusion protein is a non-limiting example of such a fusion protein, for which detailed experimental methods and results are described below. However, it is believed that other component 1 and component 2 proteins (and functional portions thereof) would form DSP fusion proteins showing at least similar behavior. Suitable component 1 and component 2 proteins are listed in Table 1.

A study was performed to demonstrate improved efficacy of TRAIL-induced apoptosis in hematological malignancies by using a fusion protein Fn14-TRAIL that combines a functional TRAIL molecule with fibroblast growth factor-inducible 14 molecule (Fn14), a receptor for TNF like weak inducer of apoptosis (TWEAK). TWEAK (TNFSF12) is another member of TNF ligands superfamily that is generally expressed as a transmembrane type II protein on a wide range of hematopoietic cell types, including macrophages, monocytes, NK cells, lymphocytes and cells of most transformed hematological lines. TWEAK was initially described as a pro-apoptotic factor for certain tumor cell lines, but subsequent studies revealed that it can stimulate many other cellular responses, including cell proliferation, survival and differentiation. Hematological malignancies generally lack Fn14 receptors and therefore are mostly resistant to signaling by TWEAK.

It was hypothesized that TRAIL, once anchored both to its death receptors and to TWEAK via the Fn14 component of fusion protein, may direct stronger pro-apoptotic signaling in malignancies. The results presented in the study demonstrate preferential binding of Fn14-TRAIL to TRAIL receptors on the malignant cells and its low pro-apoptotic capacity. However, recombinant TWEAK caused most efficient modification of Fn14-TRAIL fusion protein molecule and converted it into super TRAIL capable of inducing enhanced apoptosis in all hematological malignant lines tested and renal cell carcinoma as well.

### Experimental Procedures

Reagents

Fn14-TRAIL Fusion Protein (Fn14-TRAIL) was produced and purified to 95% purity by Cobra Bio-manufacturing (Keele, UK) from Chinese Hamster Ovary cells stably transfected with a DNA construct encoding a chimeric human Fn14-TRAIL sequence downstream of the CMV promoter. The amino acid sequence of Fn14-TRAIL fusion protein comprises the extracellular domain of human Fn14 (amino acids 1-52 of the mature protein) directly linked to extracellular domain of human TRAIL (amino acids 53-217 of the mature protein).

Soluble Recombinant Human TWEAK, Fn14 and TRAIL

Fn14 and TRAIL were purchased from PeproTech (Rocky Hill, N.J., USA). Caspases inhibitors Z-VAD-FMK, Z-IETD-FMK, and Z-LEHD-FMK (R&D Systems (Minneapolis, Minn., USA) were used in functional studies according to the manufacturer's instructions.

Cell Lines

Jurkat T-cell leukemia, two Burkitt lymphoma B-cell lines, Raji and Daudi and A498 Renal Cell Carcinoma cell line were obtained from ATCC (Bethesda, Md., USA). JY B-lymphoblastoid line was a kind gift from Prof. Mark L. Tykocinski, Jefferson Medical School, Philadelpia, Pa., USA), All lines were periodically tested for mycoplasma by using a commercial PCR kit (Biological Industries, Beit Haemek, Israel). The cells were cultured in RPMI medium, supplemented with 10% fetal calf serum, 2 mM glutamine, penicillin (100 IU/mLl), and streptomycin (100 μg/mL). All medium components were supplied by Rhenium (Modi'in, Israel).

Flow Cytometry

Immunostaining for detection of membrane-bound TRAIL, TWEAK, DR4, DR5 or Fn14 was carried out with optimal concentrations of the phycoerythrin (PE)-conjugated monoclonal antibodies (mAbs). All PE-conjugated specific mAbs and relevant isotype controls were obtained from eBioscience (San Diego, Calif., USA). Flow cytometry was performed using FACSCalibur with the CellQuest software (BD Biosciences, MD, USA). A total of 20*10³ events were counted for each sample. Data were analyzed using FCS Express 4 software (www.denovosoftware.com).

Tweak Overexpression in Jurkat Cell Line

Jurkat cell line (ATCC) cells were propagated in suspension conditions in RPMI 1640 Media supplemented with L-glutamine and 10% FBS in humidified 37° C. and 5% CO2 incubator.

106 Jurkat cells were co-transfected with 5 micro-g of ultrapure plasmid DNA in R buffer using the Jurkat microporation optimized electroporation parameters protocol (Neon™ transfection system cell protocols) of one of two expression vectors encoding for the human full length TWEAK protein using the Neon Transfection System (Invitrogen) and with GFP expressing plasmid for the assessment of the transfection efficiency. The two expression vectors differ in the mammalian promoters: pCR3.1 & pEF-DEST51, both promoting constitutive protein expression (Invitrogen). Co-transfected cells were then cultured in the above mention media and conditions. At 48 hours cells were cultivated in standard supplemented medium with the specific antibiotic selection: for pCR3.1 (1 mg/ml G-418) and for pEF-DEST51 (5 microgram/ml Blasticidin). After 3 weeks under selection pressure, 1×10⁶ cells were cultivated in supplemented regular medium for 4 days for supernatant medium collection to test the presence of the soluble form of TWEAK. The rest of the cells remained under antibiotic selection for the detection of the membrane full length TWEAK protein. The presence of membrane TWEAK in the transfected cells was confirmed by FACS using an anti-Human TWEAK antibody.

Apoptosis Induction and Detection

Indicated cells (5*10⁵) were the cultured for the indicated time in the RPMI cultivation medium containing the proteins of interest at a specified concentration. After apoptosis-inducing treatment cells were stained with annexin-V-FITC and Propidium Iodide (PI) apoptosis detection kit (MBL, Des Plaines, Ill., USA) according to the manufacturer's recommendations and analyzed using flow cytometry. A total of 20*10³ events were counted for each sample. The results were presented as average percentage±SE of all apoptotic cells (FITC⁺PI⁻ and FITC⁺PI⁺). In some cases, in apoptosis kinetics study, the percentage of cells in early apoptotic stage (FITC⁺H⁻) was also given.

Analysis of Fn14-TRAIL and TWEAK-Modified Fn14-TRAIL Binding to Jurkat Cells

Mode of Fn14-TRAIL and TWEAK-modified Fn14-TRAIL binding to target cells was compared to TRAIL binding according to expression of TRAIL and TWEAK receptors before and after incubation with the specified proteins. Jurkat cells (3*10⁶) were incubated for 30 min at 4° C. in presence of 250 ng/mL TRAIL, Fn14-TRAIL, or Fn14-TRAIL and 100 ng/mL TWEAK. Unbound proteins were removed by washing, and each cell batch, as well as untreated cells, were subdivided for immune-stained with the relevant PE-labeled antibodies and analyzed by FACS. The same experiment was repeated with cells preincubated with 80 mg/ml of DR5 neutralizing mAb.

For functional analysis of the molecules involved in apoptotic signal transmission neutralizing antibodies against Fn14, TWEAK, DR5 or TRAIL (20 mg/ml) were added to Jurkat cells (0.5*10⁶/ml) 30 min before the addition of 25 ng/ml TRAIL, Fn14-TRAIL, Fn14-TRAIL in combination with 100 ng/ml TWEAK, or TWEAK alone. Apoptosis was determined at 24 h. Neutralizing antibodies against TWEAK (clone Carl-1) were purchased from BioLegend (San Diego, Calif., USA), while neutralizing antibodies against TRAIL (RIK-2), DR5 (HS201) and Fn14 (ITEM-2), as well appropriate isotype controls were purchased from eBioscience (San Diego, Calif., USA).

### Activation of Healthy Peripheral Blood Lymphocytes (PBL) In Vitro for Evaluation of Activated T Blasts Susceptibility to Apoptosis Induction by Fn14-TRAIL and TWEAK

PBL were isolated from the blood of human volunteers using Lymphoprep™ (Axis-Shield PoC, Oslo, Norway) density gradient centrifugation according to the manufacturer's instructions. PBL (10⁶ cells/mL) were cultured for 3 days in RPMI culture medium with 0.1 mg/mL anti-CD3 (OKT-3) antibody (eBioscience (San Diego, Calif., USA) at 37° C. and 5% CO₂. Cells were resuspended in RPMI medium containing 10 EU/mL IL-2 (Roche Applied Diagnostics, Basel, Switzerland) for further cultivation (up to 16 days). At the specified time points (3, 10 and 16 days of the culture) 100 ng/mL anti-Fas mAb (CH-11, MBL, Des Plaines, Ill., USA), 250 ng/mL Fn14-TRAIL or 250 ng/mL Fn14-TRAIL combined with 100 ng/mL TWEAK were added to the culture. Apoptosis was measures at 24 h.

Whole Cell Lysates and Western Blotting

To validate protein expression, whole cell lysates were prepared by lysis of 2*10⁷ cells in 125 μl lysis buffer (100 mM NaCl, 50 mM Tris-HCl pH 8.0, 0.5% Nonidet P-40, 1 mM PMSF) supplemented with protease inhibitor and phosphatase inhibitor PhosSTOP cocktails (Roche, Basel, Switzerland). For analysis, lysates were separated on 12% or 15% SDS-polyacrylamide gels. Following electrophoresis proteins were transferred onto Immobilon-P membranes (Millipore, Billerica, Mass., USA). Immunoblotting was performed with the following antibodies: anti-caspase-8 (9746, Cell Signaling), anti-caspase-9 (LAP6, R&D systems), anti-caspase-3 (9662, Cell Signaling), anti-FLIP (NF-6, Enzo), anti-Bid (2002, Cell Signaling), anti-Rip (D94C12 XP™, Cell Signaling), anti-NFκB p65 (C-20: sc-372, Santa Cruz Biotechnology), anti-IkBα (417208, R&D systems), anti-3-phosphate dehydrogenase (GAPDH)(6C5, Millipore). Secondary species-specific antibodies coupled with horseradish peroxidase were purchased from Bio-Rad (Hercules, Calif., USA). Chemiluminescence detection kit EZ-ECL (Biological industries, Beit Haemec, Israel) was used for detection of immunospecific bands.

Western Blotting Analysis

Immunoblotting data were normalized against GAPDH using Quantity One software (Bio-RAD, Hercules, Calif., USA). The charts represent GAPDH-normalized data as average±SE of the cleaved (activated) protein fraction of total protein detected by specific Ab in the lysate. Thus p18/total caspase-8 fraction was calculated as p18/(p18+p41-43+p55) in the same well, while p34-35/total caspases-9 fraction was calculated as p34-35/(p34-35+p45) in the same well. Else, when changes in the protein expression were not accompanied by the protein cleavage, the results were normalized both against GAPDH and against untreated control. Three to five independent Western blots were analyzed for each protein.

Non-Denaturing Native Gel Electrophoresis

NativePAGE™ Novex 4-16% Bis-Tris Protein Gels and reagents were purchased from Invitrogen, and electrophoresis and Western blotting were performed according to the manufacturer's protocol. Briefly, for complex formation equimolar amounts of recombinant human sTWEAK (50 ng/μl in 50 mM Bis-Tris buffer, pH 7.2, 40 mM NaCl, 3 mM EDTA) and Fn14-TRAIL (70 ng/μl in the same buffer) were mixed and incubated 10 minutes on ice before native gel electrophoresis. Tweak, Fn14-TRAIL or their combination (2 μl) were mixed with 1 μl NativePAGE cathode additive (×20), 2.5 μl NativePAGE sample buffer (×4), and deionized water to make the total volume to 10 μl/lane. Electrophoresis was performed for 2 h at 150v at room temperature. NativeMark™ Unstained Protein Standard was used for molecular size estimation of TWEAK, Fn14-TRAIL and their complex. After gel electrophoresis, proteins were transferred to PVDF membrane for immunoblotting with anti-TRAIL antibody (Abcam) or anti-TWEAK antibody (Cell Signaling).

Statistical Analyses

The results were expressed as average percentage of 3 or 4 independent experiments and their standard error. Data were analyzed by analysis of variance to compare between the different experimental groups. P<0.05 was considered statistically significant.

### Example 1

Fn14-Trail Fusion Protein is a Weak Apoptosis Inducer in Leukemia Cell Lines

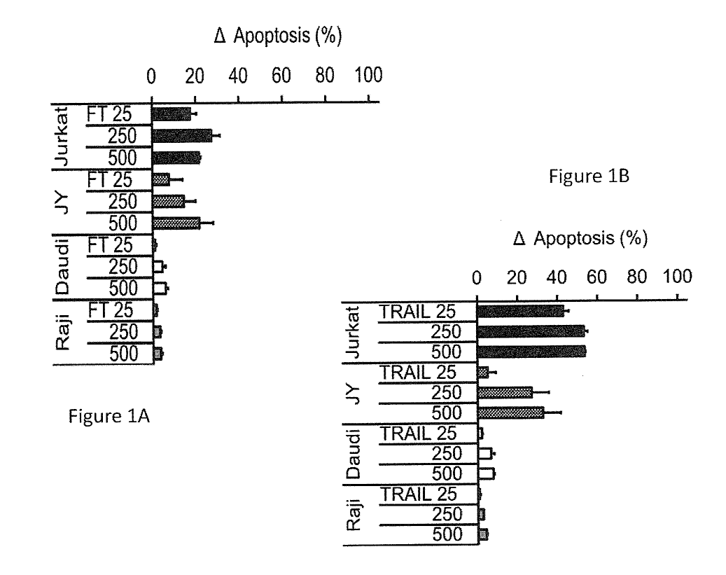

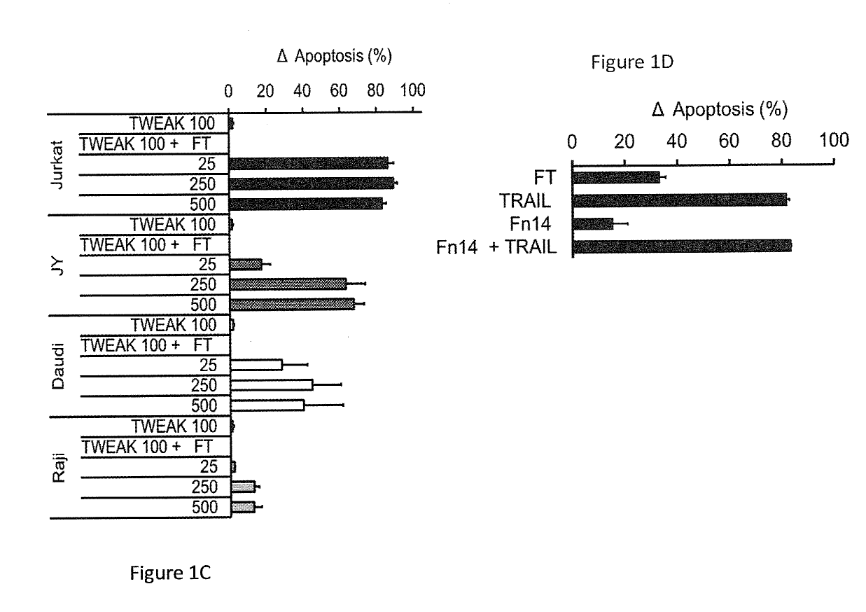

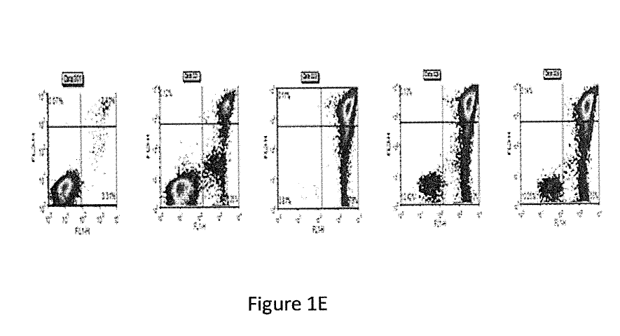

The above example considered the effect of Fn14-TRAIL on human leukemia and lymphoma cell lines (FIG. 1A). It found that incubation with Fn14-TRAIL (25 ng/mL, 250 ng/mL or 500 ng/mL) for 24 hours induced apoptosis in Jurkat T cell leukemia and in JY lymphoblastoid B cell line (maximum mortality was about 30%), while Daudi and Raji (two lines derived from Burkitt's lymphoma patients) remained resistant to apoptosis even when incubated with the highest Fn14-TRAIL dose. Prolongation of incubation time with Fn14-TRAIL to 48 h did not increase substantially cell death in vitro (data not shown).

It found that TRAIL, one of the components of the fusion protein, induces stronger apoptosis in Fn14-TRAIL-sensitive lymphoblast cell lines than Fn14-TRAIL (FIG. 1B). However, cell lines resistant to Fn14-TRAIL were also resistant to TRAIL. Using Fn14-TRAIL-sensitive Jurkat cell line the ability of Fn14-TRAIL (250 ng/ml) to induce cell death in vitro was compared with cell death caused by the same amount of the second Fn14-TRAIL component soluble Fn14 (sFn14), or to sFn14 in combination with TRAIL. FIG. 1C shows that sFn14 is unable to induce substantial apoptosis in vitro when incubated with cells alone and adds nothing to TRAIL-induced apoptosis.

### Example 2

TWEAK Significantly Potentiates Fn14-TRAIL-Induced Apoptosis of Malignant Lymphoblasts

TWEAK can efficiently bind its receptor Fn14. Therefore the experiment was conducted to assess the role that TWEAK binding may play in modification of Fn14-TRAIL-induced anti-cancer toxicity. TWEAK dose (100 ng/mL) was chosen as the dose most effective in functional studies The results presented in FIG. 1D show that cell incubation with TWEAK does not result in apoptosis of lymphoblastoid cells in vitro. However, addition of the same TWEAK dose to Fn14-TRAIL amplified apoptosis induction in all lymphoblastoid cell lines tested (compare FIGS. 1A and 1D). Apoptosis caused by Fn14-TRAIL and TWEAK combination was more profound in all the assessed cell lines i.e. Raji, Daudi, J Y and Jurkat than TRAIL-induced apoptosis (compare FIGS. 1B and 1D). Adding TWEAK to TRAIL could not augment TRAIL-induced apoptosis (FIG. 1E). These results suggest that TWEAK binding to Fn14-TRAIL specifically enhances ability of the fusion protein to induce apoptosis in cancer cells.

### Example 3

Activated Peripheral Blood Lymphocytes (PBL) from Healthy Subject are Relatively Resistant to Apoptosis by Fn14-TRAIL and TWEAK Combination

An increasing body of evidence demonstrates the physiological role of TRAIL in the regulation of immune response. TRAIL has been shown to cause apoptosis of in vitro activated human CD4+ T cell clones and PBL. Therefore the experiments examined whether combined treatment by Fn14-TRAIL plus TWEAK induces apoptosis in activated healthy PBL. Human PBL, incubated for three days in vitro with 0.1 μg/mL agonistic anti-CD3 antibody (OKT3), were cultured for 24 h with 250 ng/mL Fn14-TRAIL and 100 ng/mL TWEAK for apoptosis induction. Otherwise, OKT3-activated PBL were left in culture medium supplemented with 10 U/mL IL-2 for additional 10 or 16 days before incubation with Fn14-TRAIL and TWEAK. Susceptibility of similarly treated PBL to apoptosis induction by 100 ng/mL anti-FAS mAb served as positive control of activation. The results presented in FIGS. 14A-D show that PBL incubated three days with OKT3 mAb remained insensitive to apoptosis induction both by anti-FAS and by Fn14-TRAIL and TWEAK. Additional 10 day cultivation in IL-2-enriched medium made PBL moderately sensitive to apoptosis by Fn14-TRAIL and TWEAK (31.9%±0.03% dead cells over control) and by anti-FAS mAb (50.6%±4.8% dead cells over control). However, malignant Jurkat cells demonstrated much stronger susceptibility to Fn14-TRAIL and TWEAK and to anti-FAS (86.1%±5% and 80.9%±1.6% dead cells over control). PBL lost susceptibility to apoptosis by Fn14-TRAIL and TWEAK after sixteen day cultivation in IL-2-enriched medium, though susceptibility to apoptosis by anti-FAS remained high (59.4%±5.6%). Thus, Fn14-TRAIL and TWEAK revealed a statistically significant, moderate apoptotic effect on day 10, that subsided later.

Susceptibility of Malignant Lymphoblasts to Apoptosis Induced by Fn14-TRAIL and TWEAK Combination Correlates with a High Level of DR5.

In order to evaluate whether binding molecule profile can predict sensitivity to apoptosis by Fn14-TRAIL plus TWEAK, FACS analysis was performed for membrane-bound Fn14, and TRAIL receptors DR4 and DR5 expression on lymphoblasts of leukemia or lymphoma cell lines. FIGS. 15A-C show that Fn14 was not expressed on any of the tested lines. The percentage of cells positive for membrane-bound TRAIL receptor DR4 varied from line to line and did not correlate with the susceptibility to apoptosis. Jurkat blasts, most susceptible to apoptosis by Fn14-TRAIL and TWEAK, did not express DR4 at all. In contrast, DR5 expression on susceptible to apoptosis Jurkat and JY lymphoblasts was significantly higher (p<0.001) than DR5 expression on apoptosis resistant cells such as Daudi and Raji cells.

### Example 4

Fn14-TRAIL in the Presence of TWEAK Binds to Leukemia Cells Via DR5 Receptor

Next, the experiments assessed whether Fn14-TRAIL and Fn14-TRAIL in the presence of TWEAK (Fn14-TRAIL plus TWEAK) bind to Jurkat cells via TRAIL receptor DR5. Accordingly, Jurkat cells were incubated with neutralizing mAbs against TWEAK, sFn14, TRAIL or DR5 before treatment by Fn14-TRAIL or Fn14-TRAIL plus TWEAK for apoptosis induction. The results presented in FIG. 2 show that antibodies blocking TWEAK or Fn14 could significantly reduce cell death induced by Fn14-TRAIL plus TWEAK but were ineffective when added before administration of Fn14-TRAIL alone. On the other hand, antibodies blocking DR5 and TRAIL could considerably reduce apoptosis induced by Fn14-TRAIL alone or by Fn14-TRAIL plus TWEAK. These results suggest that Fn14-Trail fusion protein binds to Jurkat cells via DR5 receptor, while TWEAK modifies this binding.

Next the detection of DR5 receptors on cells before and after incubation with TRAIL, Fn14-TRAIL or Fn14-TRAIL plus TWEAK was tested, in conditions preventing capping of bound proteins (4° C.), by flow cytometry. Significant reduction in percentage of DR5⁺ cells was observed after Jurkat cell incubation with TRAIL or Fn14-TRAIL (FIG. 3A) suggesting binding of these proteins to the DR5 receptors. Importantly, when cells were incubated with Fn14-TRAIL plus TWEAK almost no DR5 could be detected, suggesting Fn14-TRAIL plus TWEAK exerts stronger binding to TRAIL receptors.

Another support to the assumption that Fn14-TRAIL binds to cells through its TRAIL side comes from the significant increase in the percentage of Fn14⁺ Jurkat cells following incubation with Fn14-TRAIL (FIG. 3B). This rise in percentage of Fn14⁺ cells was be prevented by pretreatment of Jurkat cells with DR5-neutralizing antibody Incubation with Fn14-TRAIL plus TWEAK caused no substantial rise in percentage of Fn14⁺ cells, possibly, because TWEAK binding to Fn14 portion of the fusion protein prevented Fn14 detection by fluorochrome-labeled antibody.

### Example 5

Binding of Fn14-TRAIL Plus TWEAK Causes Earlier and Stronger Apoptotic Death of Malignant Lymphoblasts than Fn14-TRAIL or TRAIL,

Kinetics of apoptosis induction is an important pharmacological factor influencing drug's anti-cancer efficacy. Accordingly the kinetics of apoptosis induction by Fn14-TRAIL, TRAIL, and Fn14-TRAIL plus TWEAK was compared. Robust early apoptotic effect of Fn14-TRAIL plus TWEAK was seen in Jurkat cells as early as 30 minutes after the start of incubation, and peaked at 1 h (FIG. 4B). In comparison, both Fn14-TRAIL alone or TRAIL alone induced much less apoptosis. Definite effect of the treatment was detected after 1-2 h of incubation, with a small increase in the percentage of apoptotic cells within the next 2 h (FIGS. 4A and 4C).

### Example 6

Seeking additional evidence to the assumption that cell death induced by co-culturing cells with Fn14-TRAIL plus TWEAK was due to apoptosis, by Fn14-TRAIL plus TWEAK (FIG. 4D). Moreover, addition of caspase-8 inhibitor Z-IETD-FMK and caspase-9 inhibitor Z-LEHD-FMK lead to partial blockade of apoptosis, demonstrating the contribution of both caspase pathways (intrinsic and extrinsic) in mediating cell death (FIG. 4D).

### Example 7

Addition of TWEAK to Fn14-TRAIL Induces the Formation of Large Complexes

Next the inventors investigated whether TWEAK binding to the Fn14 component of Fn14-TRAIL leads to TWEAK-Fn14-TRAIL complex formation (FIG. 5). According to the SDS-PAGE results, the Fn14-TRAIL monomer has an approximate molecular weight (MW) of 25-27.5 kDa. After BN-PAGE and immunoblot with anti-TRAIL-specific monoclonal antibody, the fusion protein was detected as a band of approximately 66 kDa (the estimated MW for the trimer is 74 kDa) and another band was seen at about 20 kDa, most relevant to the monomer size (the estimated MW of the monomer is 25 kDa). Monomer of soluble TWEAK produced by PeproTech has MW 17 kDa. After BN-PAGE and immunoblot with TWEAK-specific monoclonal antibody, it was detected as a single band of approximately 60 kDa. Anti-TWEAK and anti-TRAIL antibodies do not cross-react. Nevertheless, the mixture of equimolar amounts of Fn14-TRAIL plus TWEAK was detected by both antibodies as the same band of approximately 242 kDa (the estimated MW for the hexamer of Fn14-TRAIL+TWEAK is 250 kDa), another band that correlates to about 480 kDa was also visible together with additional larger bands. This result suggests that Fn14-TRAIL plus TWEAK form a stable complex in vitro.

### Example 8

Fn14-TRAIL Plus TWEAK Combination Boosts Activation of Intracellular Pro-Apoptotic Signaling and Inhibits Anti-Apoptotic Ones

To identify molecular mechanisms contributing to the accelerated apoptosis induction by TWEAK-modified Fn14-TRAIL, the expression kinetics of several important apoptosis-associated proteins in Jurkat cells treated by Fn14-TRAIL, TRAIL, or TWEAK-Fn14-TRAIL complex was assessed. Monitoring kinetics of caspases activation revealed that Fn14-TRAIL was rather ineffective in inducing apoptosis within time interval tested (first 90 min) Thus, expression of all examined proteins was compared between TRAIL and TWEAK-Fn14-TRAIL complex treatments (FIGS. 6, 7). We found that TWEAK-Fn14-TRAIL complex induced significantly earlier and stronger activation of caspase-8 and -9, as well as earlier processing of BID to tBID, when compared with TRAIL. TWEAK-Fn14-TRAIL complex accelerated also activation of caspase-3 and PARP. Induction of pro-apoptotic protein activation by treatment with TWEAK-Fn14-TRAIL complex was associated with diminished expression of anti-apoptotic cFLIP short, cleavage of RIP and depletion of c-IAP1 protein. Similar, but statistically insignificant, trend to diminished c-IAP2 expression was detected after treatment of cells with TWEAK-Fn14-TRAIL complex (data not shown). BCLx and XIAP proteins expression levels in cell cytosol of TWEAK-Fn14-TRAIL complex- and TRAIL-treated cells were similar (data not shown).

### Example 9

Jurkats Cells Co-Transfected with TWEAK and GFP

Jurkat (ATCC) cells were transfected with two plasmids encoding for the Human Full length TWEAK protein pCR3.1 and for pEF-DEST51 (pDEST-EF1) as described in methods. The transfection efficiency was assessed by co-transfection with a GFP plasmid (FIG. 8).

Jurkat Cells Transfected with TWEAK Show Enhanced Sensitivity to Fn14-TRAIL Apoptosis Induced Effect

After confirming the presence of TWEAK on cells' membrane by flow cytometry, the activity of the soluble and/or membrane attached forms of TWEAK was tested using the biological functional assay MTS. Based on previous data (see above), addition of exogenous TWEAK enhances the apoptosis induction ability of Fn14-TRAIL.

The survival of Jurkat naïve cells was compared to Jurkat TWEAK transfected cells after 24 hours incubation with increasing amounts of Fn14-TRAIL.

Approximately 30% reduction in cell survival was observed in naïve Jurkat cells cultured with high Fn14-TRAIL concentration (300 ng/ml). Significantly, a more pronounce effect was observed in the transfected cells. In the TWEAK transfected Jurkat cells, about 70% decrease in cell survival was observed even at much lower Fn14-TRAIL concentrations (e.g. 3 ng/ml) (FIG. 9). The difference seen between the two transfected cell lines may reside in the promoter ability of the different transfected constructs.

Incubation of the transfected and naïve Jurkat cells with 30 ng/ml Fn14-TRAIL and 30 ng/ml recombinant soluble TWEAK caused ˜100% cell death demonstrating the boosted effect of TWEAK on Fn14-TRAIL apoptosis induction.

Conditioned Media from TWEAK Transfected Cells Enhance Fn14-TRAIL Death Induced Effect

In order to test the presence of the soluble form of TWEAK, cells transfected with the human full length TWEAK gene were enriched (antibiotic selected) and cultivated in regular cell culture medium for 4 days. The supernatants were then collected and added to naïve Jurkat cells incubated in the presence of Fn14-TRAIL at 3 ng/ml and 30 ng/ml for 24 hours.

As can be seen in FIG. 10, the addition of conditioned media collected from the TWEAK gene transfected cells resulted in significantly higher (65-95%) reduction in cell survival as compared to the null effect of the supernatant collected from naïve cells or the effect of Fn14-TRAIL or recombinant soluble TWEAK (˜30-35%).

### Example 10

TWEAK Potentiates Fn14-TRAIL Inhibitory Effect on Renal Cell Carcinoma Cells Survival

Next TWEAK'S ability to potentiate Fn14-TRAIL activity in a different cell line was tested. A498 cells, a renal cell carcinoma (RCC) cell line, were incubated for 24 h with increasing amounts of Fn14-TRAIL in the presence or absence of TWEAK. As shown in FIG. 11, addition of Fn14-TRAIL plus TWEAK robustly increased the inhibitory effect of Fn14-TRAIL.

### Example 11

TWEAK Improves Fn14-TRAIL Binding to the TRAIL-Receptor DR5

After it was demonstrated that TWEAK potentiates Fn14-TRAIL activity as seen by decreased cell viability, enhanced activation of the caspases and inhibition of anti-apoptotic signals, the effect of TWEAK on Fn14-TRAIL binding to the TRAIL receptor DR5 was tested. For that, the Bia-core assay was used. Fn14-TRAIL, TWEAK or the combination of the two at different doses were loaded onto the Bia Core cheap covered with DR5. As one can see in FIG. 12, TWEAK itself does not bind to DR5. As expected, Fn14-TRAIL binds to the TRAIL receptor. Importantly, the addition of increasing amounts of TWEAK significantly augmented Fn14-TRAIL binding to DR5.

### Example 12

A498 RCC Cells Become Resistant to Fn14-TRAIL when TWEAK is Knocked Out.

To knockout the TWEAK gene, wild type A498 RCC cells were double transfected with GFP or RPF genes replacing the TWEAK gene by homologous recombination (FIG. 13A). Cells were sorted by FACS and lose of TWEAK expression was tested by immunoblotting the whole cells lysates with anti-TWEAK Abs (FIG. 13B). Wild type and TWEAK knock out cells were incubated in the presence and absence of increasing concentrations of Fn14-TRAIL for 24h. Cell's viability was estimated by MTS assay. As shown in FIGS. 13C-D, though WT A498 cells exhibited significant sensitivity to Fn14-TRAIL (FIG. 13C), the TWEAK KO cells were much more resistant FIG. 13D, however, when TWEAK was added to the cultured media (FIG. 13E), cells regained their sensitivity to Fn14-TRAIL's inhibitory effect. These results emphasize the dependence of Fn14-TRAIL on TWEAK presence for its apoptotic effect.

Overall, taken together, the data show that TWEAK binding to Fn14-TRAIL fusion protein can be regarded as a similar specific and highly effective way to improve the binding and activation of Fn14-TRAIL to the TRAIL receptors and its functional activity.

### Example 13

### Evaluation of FN14-TRAIL Activity in Murine Model Suitable for Testing Activity in Lupus/Immune Complex Nephritis

### Inducing Glomerulonephritis

The nephrotoxic serum nephritis model is a well established model of immune complex glomerulonephritis (Y. Fu, Y. Du, C. Mohan, Experimental anti-GBM disease as a tool for studying spontaneous lupus nephritis, Clin. Immunol 124 (2007) 109-118.) and is well accepted and a model to test the feasibility of different treatment regimens for lupus nephritis. In this model, renal injury is induced by passive transfer of rabbit anti-mouse glomerulus antibodies to mice pre-immunized with rabbit IgG. This results in anti-rabbit antibodies which complex with the passively transferred rabbit anti-mouse-glomerular antibodies. For that matter, glomerular isolation from C57BL/6 mice was achieved using magnetic 4.5-um diameter Dynabeads (Takemoto M et al. American Journal of Pathology, Vol. 161, No. 3, September 2002). Glomeruli were sonicated and protein was sent to rabbit immunization by Lempier LTD Pennsylvania. The nephrotoxic rabbit serum generated was then injected into primed mice (5 days after injection with 250 ug rabbit IgG (SIGMA ISRAEL) in complete Freund's adjuvant). Clinical disease is assessed via clinical signs, blood, urine and histological samples. Serum is analyzed for creatinine and blood urea nitrogen (BUN). Levels of proteinuria are determined by dipsticks and by protein:creatinine ratio Kidney damage is also be evaluated by histology by a blinded nephropathologist (Hadassah Medical Center). Glomerular mesangial proliferation, presence of glomerular crescents, tubulointerstitial changes (inflammatory infiltrate, atrophy, dilation of tubuli) are recorded and scored for level of severity. Also, immunofluoresnt staining of the kidney specimen for the evaluation of immune complexes deposits in the diseased kidneys will be tested.

### Establishing the Therapeutic Effect of Fn14-TRAIL on Nephrotoxic Glomerulonephritis: Experimental Design

Fn14-TRAIL protein will be administered to mice by s.c injections, from day 2 after Rabbit IgG injections to day 8.

In order to test the therapeutic effect of Fn14-TRAIL several control groups are planned of 10 mice each:

A. Mice injected with Citrate buffer (vehicle)
B. Mice treated with conventional immunosuppression (steroids)
C. 3 treatment groups will be included −50, 100 and 200 μg/d/mice.

Group size was determined in order to enhance ability of detecting statistical significance.

Blood and urine samples will be collected at times 0 (defined as time of injection with rabbit IgG in complete Freund's adjuvant), day 1, day 5 (injection of nephrotoxic rabbit serum), day 7, day 14 and day 21.

Kidneys at various time points (pending clinical parameters, most probably on days 14, 21, 28) will be harvested and preserved for frozen section and immuno-florescence (IgG, IgM, IgA, C3, C4, albumin) as well as preservation in paraffin sections for light microscopy examination. Kidney samples will also be kept for future RNA and DNA testing.

Our working hypothesis is that treatment with Fn14-TRAIL will attenuate clinical and histological parameters of glomerulonephritis. Treatment groups are expected to have a smaller rise in serum creatinine and urea, and less histological damage (as will be reflected in less crescent formation, inflammatory infiltrate tubulointerstitial damage and less immune-complexes deposits). For evaluating the effectiveness of the treatment regimens, serum samples will be taken at different time points during the treatment to measure Fn14-TRAIL serum concentrations.

### Example 14

The experiments detailed in Example 2 and Example 10 is repeated with the following compositions comprising fusion proteins having the ECD of Fn14 and FasL, CD40L, RANKL, 41BBL, TNF-ALPHA, wherein TWEAK is added to the composition:

The expected results are that the presence of TWEAK will enhance the apoptosis in comparison to the apoptosis caused the addition of the fusion protein alone.

### Example 15

Another example for possible cluster forming protein according to the embodiment of the invention.

### A-CD40L

### B-CD40

### C-FasL

D-Fas (the receptor for FasL, CD95)

The amino acids of the ECD of CD40 (1-193) were fused to the ECD of FasL (127-281) to form CD40-FasL. In order to test whether in the presence of CD40L (first ligand also termed ligand A) or in the presence of Fas (second receptor also termed receptor D) CD40L or Fas will be added to a medium containing a fusion protein CD40-FasL at different rations for 2:0.5 to 1:5 (CD40-FasL to added fusion protein) and the proteins will be separated on a native gel as described above for the combination of Fn14-trail plus tweak. The resulting gel will be blotted with all of the following—anti CD40 Abs, anti CD40L Abs, anti FasL Abs and anti Fas Abs. The existence of larger complexes than a trimer will support the formation of a cluster.

In order to test the importance of CD40L for CD40-FasL activity, cells that are positive to Fas (and therefor are sensitive to the apoptotic effect of the FasL domain of the protein) but are negative for CD40L will be incubated with CD40-FasL in the presence or absence of exogenous CD40L. Cell survival will be estimated using MTS assay, and apoptosis induction will be assessed be Annexin/PI staining and FACS analysis. Other sets of experiments will be performed on Fas positive and CD40L positive cells that are highly sensitive to CD40-FasL apoptotic effect in the presence of absence of blocking antibodies against CD40L that will prevent it from binding the CD40-FasL protein and therefor will prevent the formation of a cluster. Also, cultured medium of cells positive and negative for CD40L will be incubated with CD40-FasL and the presence of clusters will be evaluated using native page gel as described above.

While the invention has been described with respect to a limited number of embodiments, it will be appreciated that many variations, modifications and other applications of the invention may be made, and that various combinations and subcombinations of embodiments are also possible and encompassed within the scope of this application.

1. A method of treating a disease in a subject, wherein the disease is associated with presence or high level of a first TNF-family ligand in comparison to a healthy subject, as measured in a diseased tissue or organ or in the envornment or in the blood, Cerebrospinal Fluid (CSF), synovial fluid, saliva, or urine or any other body fluids or excretions of the subject, the method comprising: administering a fusion protein to the subject to treat the disease, the fusion protein comprising an extracellular domain (ECD) of a first TNF-family receptor, wherein the fusion protein further comprises a second TNF-family ligand, wherein the second TNF-family ligand is not capable of binding to the first receptor but wherein the first TNF-family ligand is capable of binding to the first TNF-family receptor thereby treating the disease in the subject.

2. A method of treating a disease in a subject, comprising: administering a fusion protein to the subject to treat the disease, the fusion protein comprising an extracellular domain (ECD) of a first TNF-family receptor, wherein the fusion protein further comprises a second TNF-family ligand, wherein the second TNF-family ligand is not capable of binding to the first receptor but wherein the second TNF-family ligand is capable of binding to a second TNF-family receptor, wherein the disease is treatable by activating the second TNF-family receptor.

3. A method of treating a disease in a subject, comprising: determining a presence or a level of a first TNF-family ligand capable of binding to a first TNF-family receptor in the subject; if said level of said first ligand is above a baseline level, administering a fusion protein to the subject to treat the disease, the fusion protein comprising an extracellular domain (ECD) of said first TNF-family receptor, wherein said fusion protein further comprises a second TNF-family ligand, wherein said second TNF-family ligand is not capable of binding to said first receptor but wherein said second TNF-family ligand is capable of binding to a second TNF-family receptor, wherein said disease is treatable by activating said second TNF-family receptor.

4. The method of claim 3 further comprising detecting a level of a second TNF-family receptor, to which said second TNF-family ligand is capable of binding, in the subject; and administering said fusion protein after detecting said level of said second TNF-family receptor in said subject.

5. The method of claim 2, wherein the first and the second receptor and the first and the second ligand are selected as follows:
if the first receptor is 4-1BB, the first ligand is 4-1BBL; or wherein if the second receptor is 4-1BB, the second ligand is 4-1BBL;
if the first receptor is BCMA, the first ligand is APRIL or BAFF; or wherein if the second receptor is BCMA, the second ligand is APRIL or BAFF;
if the first receptor is CD27, the first ligand is CD27L; or wherein if the second receptor is CD27, the second ligand is CD27L;
if the first receptor is CD30, the first ligand is CD30L; or wherein if the second receptor is CD30, the second ligand is CD30L;
if the first receptor is CD40, the first ligand is CD40L; or wherein if the second receptor is CD40, the second ligand is CD40L;
if the first receptor is EDAR, the first ligand is EDA-A1; or wherein if the second receptor is EDAR, the second ligand is EDA-A1;
if the first receptor is XEDAR, the first ligand is EDA-A2; or wherein if the second receptor is XEDAR, the second ligand is EDA-A2;
if the first ligand is TRAIL, the first receptor is selected from the group consisting of TRAIL-R1, TRAIL-R2, TRAIL-R3 and TRAIL-R4; or wherein if the second ligand is TRAIL, the second receptor is selected from the group consisting of TRAIL-R1, TRAIL-R2, TRAIL-R3 and TRAIL-R4;
if the first ligand is TRANCE/RANKL, the first receptor is selected from the group consisting of OPG and RANK; or wherein if the second ligand is TRANCE/RANKL, the second receptor is selected from the group consisting of OPG and RANK;
if the first receptor is TROY, the first ligand is TROY ligand; or wherein if the second receptor is TROY, the second ligand is TROY ligand;
if the first receptor is Fas, the first ligand is FasL; or wherein if the second receptor is Fas, the second ligand is FasL; or
if the first receptor is GITR, the first ligand is GITL; or wherein if the second receptor is GITR, the second ligand is GITL.
if the first ligand is LIGHT, the first receptor is selected from the group consisting of DcR3 and HVEM; or wherein if the second ligand is LIGHT, the second receptor is selected from the group consisting of DcR3 and HVEM.
if the first receptor is DR3, the first ligand is TL1A/VEGI; or wherein if the second receptor is DR3, the second ligand is TL1A/VEGI.
if the first receptor is Fn14, the first ligand is TWEAK; or wherein if the second receptor is Fn14, the second ligand is TWEAK.
if the first receptor is TNFR1, the first ligand is TNF-alpha; wherein if the second receptor is TNFR1, the second ligand is TNF-alpha.
if the first receptor is TNFR2, the first ligand is TNF-beta; or wherein if the second receptor is TNFR2, the second ligand is TNF-beta.
if the first receptor is Lymphotoxin beta R, the first ligand is selected from the group consisting of LIGHT, lymphotoxin alpha (LTA), and lymphotoxin beta (LTB); or wherein if the second receptor is Lymphotoxin beta R, the second ligand is selected from the group consisting of LIGHT, lymphotoxin alpha (LTA), and lymphotoxin beta (LTB).
if the first receptor is OX40R, the first ligand is OX40L; or wherein if the second receptor is OX40R, the second ligand is OX40L.
if the first receptor is NGFR, the first ligand is selected from the group consisting of NGF and NTF4; or wherein if the second receptor is NGFR, the second ligand is selected from the group consisting of NGF and NTF4.
if the first receptor is DR6, the first ligand is APP; or wherein if the second receptor is DR6, the second ligand is APP.
if the first receptor is RELT, the first ligand is RELT ligand; or wherein if the second receptor is RELT, the second ligand is RELT ligand.

6. The method of claim 2, adapted for treatment of cancer.

7. The method of claim 2, wherein the first receptor is Fn14, the first ligand is TWEAK and the second ligand is TRAIL.

8. The method of claim 7, wherein the disease is selected from Seborrheic keratosis, Inflammatory bowel disease (IBD), such as, ulcerative colitis and Crohn's disease, Lupus Nephritis, Parkinson's disease, amyotrophic lateral sclerosis (ALS), psoriasis vulgaris, psoriatic arthritis, myocardial infarction, proliferative diabetic retinopathy, or retinopathy caused by any other condition, such as hypertension, radiation, sickle cell disease and the like), or acute ischemic stroke.

9. The method of claim 7, wherein the disease is selected from testis cancer, urothelial cancer, Hodgkin lymphoma, squamous cell carcinoma, keratinocyte carcinoma, or osteosarcoma.

10. A stabilized composition comprising a fusion protein and a first ligand in a ratio sufficient to increase therapeutic efficacy of the fusion protein in a subject, the fusion protein comprising an extracellular domain (ECD) of a first receptor and a second ligand, wherein the second ligand is capable of binding to a second receptor, wherein the first ligand is capable of binding to the first receptor, wherein the first and second ligands, and the first and second receptors, are TNF-family members, and wherein the first and second ligands are different, and the first and second receptors are different.

11. (canceled)

12. (canceled)

13. (canceled)

14. (canceled)

15. (canceled)

16. The method of claim 2, wherein the disease is a fibrotic disease.

17. The method of claim 16, wherein the disease is liver fibrosis, kidney fibrosis and/or lung fibrosis.

18. The method of claim 2, wherein the disease is liver fibrosis and the fusion protein is Fn14-TRAIL.
